(19) United States
(12) Patent Application Publication
(10) Pub. No.: US 2010/0158846 A1
(43) Pub. Date: Jun. 24, 2010

(54) HAIR-BINDING PEPTIDES

(75) Inventors: STEPHEN R. FAHNESTOCK, Wilmington, DE (US); Eberhard Schneider, Denkte (DE); Gregor Schurmann, Hannover (DE); Peter Wagner, Braunschweig (DE)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, Wilmington, DE (US)

(21) Appl. No.: 12/629,068

(22) Filed: Dec. 2, 2009

Related U.S. Application Data
(60) Provisional application No. 61/138,636, filed on Dec. 18, 2008.

(51) Int. Cl. A61K 8/64; C07K 14/00; A61Q 5/10

(52) U.S. Cl. 424/70.6; 530/324

(57) ABSTRACT

Hair-binding peptides were isolated for their use in a variety of personal care formulations and applications. The isolation of hair-binding peptides was accomplished by enrichment using mRNA-display selection technology. Hair care compositions comprising peptide-based reagents prepared comprising the hair-binding peptides are also provided.

### CROSS-REFERENCE TO A RELATED APPLICATION

This application claims the benefit of U.S. Provisional Patent Application No. 61/138,636 filed Dec. 18, 2008, incorporated herein by reference.

### FIELD

The invention relates to the field of personal care products. More specifically, the invention relates to hair-binding peptides and peptide-based hair reagents comprising hair-binding peptides.

### BACKGROUND

Proteinaceous materials having strong affinity for a body surface have been used for targeted delivery of one or more personal care benefit agents. However, many of these materials used for targeted delivery are comprised or derived from immunoglobulins or immunoglobulin fragments (antibodies, antibody fragments, Fab, single-chain variable fragments (scFv), and Camelidae VHH) having affinity for the target surface. For example, Horikoshi et al. in JP 08104614 and Igarashi et al. in U.S. Pat. No. 5,597,386 describe hair coloring agents that consist of an anti-keratin antibody covalently attached to a dye or pigment. The antibody binds to the hair, thereby enhancing the binding of the hair coloring agent to the hair. Similarly, Kizawa et al. in JP 09003100 describe an antibody that recognizes the surface layer of hair and its use to treat hair. A hair coloring agent consisting of that anti-hair antibody coupled to colored latex particles is also described. The use of antibodies to enhance the binding of dyes to the hair is effective in increasing the durability of the hair coloring, but the antibodies are difficult and expensive to produce. Terada et al. in JP 2002363026 describe the use of conjugates consisting of single-chain antibodies, preferably anti-keratin, coupled to dyes, ligands, and cosmetic agents for skin and hair care compositions. Although single-chain antibodies may be prepared using genetic engineering techniques, these molecules are expensive to prepare and may not be suitable for use in commercial personal care products due to their conserved structure and large size.

Non-immunoglobulin-derived scaffold proteins have also been developed for targeted delivery of benefit agents to a target surface, such as delivery of cosmetic agents to keratin-containing materials (See Binz, H. et al. (2005) Nature Biotechnology 23, 1257-1268 for a review of various proteins used in scaffold-assisted binding). Findlay in WO 00/048558 describes the use of calycin-like scaffold proteins, such as β-lactoglobulin, which contain a binding domain for a cosmetic agent and another binding domain that binds to at least a part of the surface of a hair fiber or skin surface, for conditioners, dyes, and perfumes. Houtzager et al. in WO 03/050283 and US 2006/0140889 also describe affinity proteins having a defined core scaffold structure for controlled application of cosmetic substances. As with immunoglobulin-like proteins, these large scaffold protein are somewhat limited by the requirement to maintain the underlying core structure for effective binding and are expensive to produce.

Target surface-binding peptides having strong affinity for a target surface have been identified and isolated from peptide libraries using any number of biopanning techniques including, but not limited to bacterial display (Kemp, D. J.; Proc. Natl. Acad. Sci. USA 78(7): 4520-4524 (1981); yeast display (Chien et al., Proc Natl Acad Sci USA 88(21): 9578-82 (1991)), combinatorial solid phase peptide synthesis (U.S. Pat. No. 5,449,754; U.S. Pat. No. 5,480,971; U.S. Pat. No. 5,585,275 and U.S. Pat. No. 5,639,603), phage display (U.S. Pat. No. 5,223,409; U.S. Pat. No. 5,403,484; U.S. Pat. No. 5,571,698; and U.S. Pat. No. 5,837,500), ribosome display (U.S. Pat. No. 5,643,768; U.S. Pat. No. 5,658,754; and U.S. Pat. No. 7,074,557), and mRNA display technology (PROFUSION™; U.S. Pat. No. 6,258,558; U.S. Pat. No. 6,518,018; U.S. Pat. No. 6,281,344; U.S. Pat. No. 6,214,553; U.S. Pat. No. 6,261,804; U.S. Pat. No. 6,207,446; U.S. Pat. No. 6,846,655; U.S. Pat. No. 6,312,927; U.S. Pat. No. 6,602,685; U.S. Pat. No. 6,416,950; U.S. Pat. No. 6,429,300; U.S. Pat. No. 7,078,197; and U.S. Pat. No. 6,436,665). Techniques to generate random peptide libraries are described in Dani, M., J. of Receptor & Signal Transduction Res., 21(4):447-468 (2001). Phage display libraries are available commercially from companies such as New England BioLabs (Beverly, Mass.).

Single chain peptide-based reagents lacking a scaffold support or immunoglobulin fold have been developed that can be used to couple benefit agents to a target surface. Examples of target surfaces include, but not are limited to body surfaces such as hair, skin, nail, and teeth (U.S. Pat. Nos. 7,220,405; 7,309,482; and 7,285,264; U.S. Patent Application Publication NOs. 2005/0226839; 2007/0196305; 2006/0199206; 2007/0065387; 2008/0107614; 2007/0110686; and 2006/0073111; and published PCT applications WO2008/054746; WO2004/048399, and WO2008/073368) as well as other surfaces such as pigments and miscellaneous print media (U.S. Patent Application Publication No. 2005/0054752), and various polymers such as polymethylmethacrylate (U.S. Patent Application Publication No. 2007/0265431), polypropylene (U.S. Patent Application Publication No. 2007/0264720), nylon (U.S. Patent Application Publication Nos. 2007/0141629 and 2003/0185870), polytetrafluoroethylene (U.S. patent application Ser. No. 11/607,734), polyethylene (U.S. Patent Application Publication No. 2007/0141628), and polystyrene (U.S. Patent Application Publication No. 2007/0261775). However, some single chain peptide-based reagents may lack the durability required for certain commercial applications, especially when coupling a particulate benefit agent to a body surface in a highly stringent matrix.

The problem to be solved is to provide additional hair-binding peptides having strong affinity for hair as well as peptide reagents comprising such hair-binding peptides for delivery of a benefit agent to the hair surface.

### SUMMARY

The invention provides sequences of peptides that bind with high affinity to hair. In one embodiment, a hair-binding peptide is provided having an amino acid sequence selected from the group consisting of: of SEQ ID NOs: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, and 54.

In another embodiment, a peptide-based reagent is also provided, said peptide-based reagent comprising the general formula:

(HBP)ₙ—BA

or

[(HBP)ₘ—S]ₙ—BA

wherein;
a) HBP is at least one of the present hair-binding peptide;
b) BA is a benefit agent;
c) S is a spacer;
d) m ranges from 1 to about 50; and
e) n ranges from 1 to about 1,000.
   wherein the hair-binding peptide has a sequence selected from the group consisting of SEQ ID NOs: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, and 54. The benefit agent may be a particulate benefit agent. As such, a peptide-based reagent is provided comprising the general structure:

[(HBP)ₘ—(BABP)ₙ]ₓ

or

[[(HBP)ₘ—S_{q}]ₓ-[(BABP)ₙ—Sᵣ]_{z}]_{y},
a) HBP is at least one of the present hair-binding peptides;
b) BABP is a benefit agent-binding peptide;
c) S is a molecular spacer; and
wherein m, n, x and z independently range from 1 to about 10, y is from 1 to about 5, and where q and r are each independently 0 or 1, provided that both r and q may not be 0.

In a further embodiment, a hair care composition comprising an effective amount of at least one of the present hair-binding peptides or peptide-based reagents is also provided.

In another embodiment, a method of delivery a benefit agent to a hair surface is provided comprising:

a) providing a hair care composition comprising at least one of the present peptide-based reagents and at least one benefit agent;

b) contacting hair with the hair care composition of (a) whereby the peptide-based reagent couples the benefit agent to hair.

The peptide-based reagents may be used to form a protective layer on a hair surface. In one embodiment, a method to form protective layer on the surface of hair is provided comprising:

a) providing a hair-care composition comprising at least one of the present hair-binding peptides;

b) contacting hair with an effective amount of the hair-care composition of (a) whereby the peptide-based reagent adheres to hair.

### BRIEF DESCRIPTION OF THE BIOLOGICAL SEQUENCES

The following sequences conform with 37 C.F.R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37C.F.R. §1.822.

SEQ ID NO: 1 is the amino acid sequence of a constant N-terminal flaking region comprising a hexa-histidine tag and a flexible linker.

SEQ ID NO: 2 is the amino acid sequence of

SEQ ID NO: 3 is the amino acid sequence of a C-terminal constant region comprising a flexible linker region and a C-terminal sequence optimized for efficient coupling to an MHA-oligonucleotide linker.

SEQ ID NOs: 4-5 are PCR primers.

SEQ ID NOs: 6-54 are hair-binding peptides biopanned against gray hair.

SEQ ID NO: 55 is the amino acid sequence of the Caspase-3 cleavage sequence.

SEQ ID NOs: 56-114 are the amino acid sequence of polymer-binding peptides.

SEQ ID NOs: 115-118 are the amino acid sequence of various cellulose acetate-binding peptides.

SEQ ID NOs: 119-173 are the amino acid sequences of various pigment-binding peptides.

SEQ ID NOs: 174-188 are the amino acid sequence of clay-binding peptides.

SEQ ID NOs: 189-214 are the amino acid sequences of calcium carbonate-binding peptides.

SEQ ID NOs: 215-237 are the amino acid sequences of silica-binding peptides.

SEQ ID NOs: 238-266 are the amino acid sequences of antimicrobial peptides.

SEQ ID NOs: 267-268 are the amino acid sequences of several peptide linkers.

SEQ ID NOs: 269-270 are the amino acid sequences of several peptide bridges.

SEQ ID NO: 271 is the amino acid sequence of peptide CXH-G2 used in Example 2.

### DETAILED DESCRIPTION OF THE INVENTION

Novel gray hair-binding peptides having strong affinity for gray hair have been identified using mRNA-display comprising an amino acid sequence selected from the group consisting of SEQ ID NOs: 6-54. Also provided are hair-binding domains formed by linking together of at least two of the present hair-binding peptides In one embodiment, a first hair-binding peptide and a second hair-binding peptide are separated by at least one linker, wherein the first and the second hair-binding peptide may be the same or different. In another embodiment, the spacer/linker is a peptide linker. In a further embodiment the peptide linker is a rigid peptide linker.

The present hair-binding peptides can be used to prepare peptide-based reagents having affinity for a hair surface. The peptide reagents may comprise a plurality of the present hair-binding peptides coupled together to form a hair-binding hand. The present peptide reagents may also comprise at least one portion capable of being coupled to a benefit agent. In one embodiment, a portion of the peptide reagent comprises a benefit agent-binding domain.

The following definitions are used herein and should be referred to for interpretation of the claims and the specification. Unless otherwise noted, all U.S. patents and U.S. patent applications referenced herein are incorporated by reference in their entirety.

As used herein, the articles “a”, “an”, and “the” preceding an element or component of the invention are intended to be nonrestrictive regarding the number of instances (i.e., occurrences) of the element or component. Therefore “a”, “an” and “the” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “about” modifying the quantity of an ingredient or reactant of the invention or employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein, the term “invention” or “present invention” as used herein is a non-limiting term and is not intended to refer to any single embodiment of the particular invention but encompasses all possible embodiments as described in the specification and the claims.

As used herein, the terms “polypeptide” and “peptide” will be used interchangeably to refer to a polymer of two or more amino acids joined together by a peptide bond. In one aspect, this term also includes post expression modifications of the polypeptide, for example, glycosylations, acetylations, phosphorylations and the like. Included within the definition are, for example, peptides containing one or more analogues of an amino acid or labeled amino acids and peptidomimetics. In one embodiment, the peptides are comprised of L-amino acids.

As used herein, the term “hair” as used herein refers to human hair, eyebrows, and eyelashes. The term “hair surface” will mean the surface of human hair capable of binding to one of the present hair-binding peptides. As used herein, the term “hair-binding peptide” (HBP) refers to at least one of the present peptide sequences that bind with high affinity to hair, such as gray hair.

As used herein, the term “benefit agent’ is a general term applying to a compound or substance that may be coupled with a hair-binding peptide for application to a hair. Benefit agents typically include conditioners, colorants, fragrances, bleaching agents, and the like along with other substances commonly used in the personal care industry. The benefit agent may be a particulate benefit agent, such as a pigment.

As used herein, the terms “coupling” and “coupled” refer to any chemical association and includes both covalent and non-covalent interactions. In one embodiment, the coupling is non-covalent. In another embodiment, the coupling is covalent.

As used herein, the term “stringency” as it is applied to the selection of the hair-binding peptides of the present invention, refers to the concentration of the eluting agent (usually detergent) used to elute peptides from the hair surface. Higher concentrations of the eluting agent provide more stringent conditions.

As used herein, the terms “hair hand” and “hair-binding domain” will refer to a single chain peptide comprising of at least two hair-binding peptides linked together by an optional molecular spacer, wherein the inclusion of a molecular spacer is preferred. In one embodiment, the molecular spacer is a peptide linker.

As used herein, the term “peptide-based reagent” or “peptide reagent” refers to a single chain peptide comprising at least one of the present hair-binding peptides. In one embodiment, the peptide-based reagent comprises two or more of the present hair-binding peptides separated by a molecular spacer. In a further embodiment, the peptide-based reagent comprises at least two of the present hair-binding peptides separated by a peptide linker. The peptide-based reagent may also have at least one region that can be coupled to the benefit agent. As such, the peptide-based reagent is used as an interfacial material to couple a hair benefit agent to the surface on human hair. The benefit agent-binding region may be comprised of at least benefit agent-binding peptide. In one embodiment, a benefit agent-binding domain is included by linking together 2 or more benefit-agent binding peptides, preferably with one or more peptide linkers.

As used herein, the term “benefit agent-binding hand” or “benefit agent-binding domain” will refer to a single chain peptide domain comprising two or more benefit agent-binding peptides (BABPs) coupled together by at least one peptide linker.

As used herein, a “polymer” is a natural or synthetic compound of usually high molecular weight consisting of repeated linked units.

As used herein, the term “pigment” means an insoluble colorant. A wide variety of organic and inorganic pigments alone or in combination may be used in the present invention. As used herein, the term “pigment lake” or “lake” refers to a pigment manufactured by precipitating a dye with an inert binder, usually a metallic salt.

As used herein, “PBP” means pigment-binding peptide. Pigment-binding peptides have been reported in the art (U.S. Patent Application Publ. No. 2005-0054752 and U.S. Pat. No. 7,285,264) and are provided as SEQ ID NOs: 119-173. SEQ ID NOs: 144-173 bind to iron oxide-based pigments.

As used herein, “PMBP” means polymer-binding peptide. As used herein, the term “polymer-binding peptide” refers to peptide sequences that bind with high affinity to a specified polymer (U.S. patent application Ser. No. 11/516,362). Examples include peptides that bind to poly(methyl methacrylate) (SEQ ID NOs: 56-82), polypropylene (SEQ ID NOs: 83-89), polytetrafluoroethylene (SEQ ID NOs: 90-98), polyethylene (99-105), nylon (SEQ ID NOs: 106-110), and polystyrene (SEQ ID NOs: 112-114).

As used herein, the term “cellulose acetate-binding peptide” is a peptide that binds with high affinity to cellulose acetate. Examples of cellulose acetate-binding peptides are provided as SEQ ID NOs: 115-118.

As used herein, “SiBP” mean silica-binding peptide. Examples of silica-binding peptides are provided as SEQ ID NOs: 215-237.

As used herein, “clay-binding peptide” refers to a peptide that binds with high affinity to clay (U.S. patent application Ser. No. 11/696,380). Examples of clay-binding peptides are provided as SEQ ID NOs: 174-188.

As used herein, “calcium carbonate-binding peptide” refers to a peptide that binds with high affinity to calcium carbonate (U.S. patent application Ser. No. 11/828,539). Examples of calcium carbonate-binding peptides are provided as SEQ ID NOs: 189-214.

As used herein, an “antimicrobial peptide” is a peptide having the ability to kill microbial cell populations (U.S. patent application Ser. No. 11/516,362). Examples of antimicrobial peptides are provided as SEQ ID NOs: 238-266.

As used herein, the term “operably-linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). In a further embodiment, the definition of “operably linked” may also be extended to describe the products of chimeric genes.

As used herein, the “benefit agent” or “hair benefit agent” refers to a molecule that imparts a desired functionality or benefit when applied or coupled to a hair surface. The present single chain peptide-based reagents may be used to couple a benefit agent to hair. In one embodiment, the peptide reagent is used to couple a benefit agent to a hair surface by forming a complex between the peptide-based reagent, the benefit agent, and the hair surface. In one embodiment, the peptide-based reagent is applied to the hair surface prior to the application of the benefit agent (i.e., a sequential application). In another embodiment, the peptide reagent and the benefit agent is applied to the hair surface concomitantly. The benefit agent may be a peptide or the peptide reagent (e.g., condition peptides or antimicrobial peptides) or may be one or more molecules bound to (covalently or non-covalently) or associated with a peptide reagent having affinity for a hair surface. The benefit agent may be a particulate benefit agent. In one embodiment, the term “particulate benefit agent’ is a general term relating to a particulate substance, which when applied to a hair surface provides a cosmetic or prophylactic effect. Particulate benefit agents typically include pigments, particulate conditioners, inorganic sunscreens and the like, along with other particulate substances commonly used in the hair care industry.

The particulate benefit agent may comprise an applied coating, such as a polymeric coating or a silica coating. Examples of benefits agents may include, but are not limited to conditioners for personal care products, pigments, dyes, fragrances, ultraviolet light blocking agents (i.e., active agents in sunscreen protectants), and antimicrobial agents (e.g., antimicrobial peptides), to name a few. In a preferred aspect, the benefit agent is cosmetically acceptable pigment or coated pigment.

As used herein, the term “MB₅₀” refers to the concentration of the binding peptide that gives a signal that is 50% of the maximum signal obtained in an ELISA-based binding assay (see Example 9 of U.S. Published Patent Application No. 2005-0226839; hereby incorporated by reference). The MB₅₀ value provides an indication of the strength of the binding interaction or affinity of the components of the complex. The lower the MB₅₀ value, the stronger the interaction of the peptide with its corresponding substrate.

As used herein, the terms “binding affinity” or “affinity” refers to the strength of the interaction of a binding peptide (e.g. target surface-binding peptides, target surface-binding domains, and peptide reagents) with its respective substrate. The binding affinity may be reported in terms of the MB₅₀ value as determined in an ELISA-based binding assay or as a K_{D} (equilibrium dissociation constant) value, which may be deduced using surface plasmon resonance (SPR).

As used herein, the term “strong affinity” refers to a binding affinity, as measured as an MB₅₀ of K_{D} value, of 10⁻⁴ M or less, preferably less than 10⁻⁵ M, more preferably less than 10⁻⁶ M, more preferably less than 10⁻⁷ M, even more preferably less than 10⁻⁸ M, and most preferably less than 10⁻⁹ M.

As used herein, “S” means molecular spacer. The spacer may be a peptide or non-peptide-based spacer. In one embodiment, the spacer is a “peptide spacer”. Depending upon elements with the peptide-based reagent being linked together the peptide spacer may also be referred to as a peptide “linker” (i.e., when linking together 2 or more target surface-binding peptides or “fingers” to form a binding “hand”) or a peptide “bridge” (i.e., a peptide spacer used to link/bridge a hair-binding hand to a peptide domain capable of binding to the surface of a particulate benefit agent).

As used herein, the terms “peptide linker” will refer to a peptide used to link together two or more target surface-binding peptides (“fingers”). In one embodiment, the peptide linker is 1 to 60 amino acids in length, preferably 3 to 50 amino acids in length. Examples of peptide linkers are provided as SEQ ID NOs: 267-268.

As used herein, the term “peptide finger” will be used to refer to an individual target surface-binding peptide, typically identified by biopanning against a target surface.

As used herein, the term “peptide hand” will be used to refer to a target surface-binding domain or region comprising 2 or more “fingers” coupled together using an optional peptide linker, wherein the inclusion of a peptide linker is preferred. As used herein, the term “bridge”, “peptide bridge”, and “bridging element” will refer to a linear peptide used to join a hair-binding domain (“hair-binding hand”) to a peptide domain capable of binding to the surface of particulate benefit agent (i.e., covalent or non-covalent coupling). The peptide bridge may range in size from 1 to 60 amino acids in length, preferably 6 to 30 amino acids in length. Examples of peptide bridges are provided as SEQ ID NOs: 269-270.

As used herein, the term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Miscellaneous (or as defined herein) | Xaa | X |

As used herein, the term “PCR” or “polymerase chain reaction” refers to a technique used for the amplification of specific DNA segments (U.S. Pat. Nos. 4,683,195 and 4,800,159).

As used herein, the term “peptide-based” refers to an interfacial material comprised of a compound pertaining to or having the nature or the composition of the peptide class. Interfacial refers to the quality of the peptide-based material described herein as connecting one material to another.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

As used herein, the term “expression”, as used herein, refers to the process by which a gene's coded information is converted into the structures present and operating in the cell. Expressed genes include those that are transcribed into mRNA and then translated into protein and those that are transcribed into RNA but not translated into protein (e.g., transfer and ribosomal RNAs). Expression may also refer to translation of mRNA into a polypeptide.

As used herein, the term “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

As used herein, the term “host cell” refers to cell which has been transformed or transfected, or is capable of transformation or transfection by an exogenous polynucleotide sequence.

As used herein, the terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

As used herein, the term “mRNA display” is an in vitro selection technique used to obtain peptides affinity that have an affinity for a target ligand/material from libraries of diverse sequences of peptides and proteins (U.S. Pat. No. 6,258,558). The process relies on mRNA-protein fusion molecules, which consist of peptide or protein sequences covalently linked via their C-termini to the 3′ end of their own mRNA (these molecules are commercially referred to as PROFUSION™ molecules; Adnexus Therapeutics, Weltham, Mass.). The library of PROFUSION™ molecules is preferably subjected to reverse transcription (i.e. transcribed into a library of DNA/RNA-protein fusion molecules) prior affinity selection. The library of fusion molecules is subjected to repetitive rounds of in vitro selection in the presence of target (typically a solid or a material immobilized on a solid support). A series of washing steps are used to select the fusion molecules exhibiting an affinity for the target material. The stringency of the washing is adjusted to select the fusion molecules those with the highest affinity (the affinity of the fusion molecule for the target material is attributed to the specific peptide sequence displayed). Selected fusion molecules are then subsequently subjected to PCR amplification. The end result is a pool of nucleotide sequences encoding peptides which have an affinity for the target ligand. The process is typically repeated for several cycles and may also include mutagenesis (e.g. error prone PCR) to evolve and identify proteins having improved affinity for the target ligand.

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

### Binding Affinity

The present hair-binding peptides exhibit a strong affinity for hair based on their ability to bind to hair after many rounds of selection under stringent conditions. The affinity of the peptide for the hair can be expressed in terms of the dissociation constant K_{D} value or an ELISA-based MB₅₀ value. K_{D} (expressed as molar concentration) corresponds to the concentration of peptide at which the binding site on the target is half occupied, i.e., when the concentration of target with peptide bound (bound target material) equals the concentration of target with no peptide bound. The smaller the dissociation constant, the more tightly bound the peptide is; for example, a peptide with a nanomolar (nM) dissociation constant binds more tightly than a peptide with a micromolar (μM) dissociation constant. In one embodiment, the present hair-binding peptides have a K_{D} value of 10⁻⁴ M or less, preferably 10⁻⁵ M or less, more preferably 10⁻⁶ M or less, even more preferably 10⁻⁷ M or less, yet even more preferably 10⁻⁸ M or less, and most preferably 10⁻⁹ M or less.

Alternatively, one of skill in the art can also use an ELISA-based assay to calculate a relative affinity of the peptide for the target material (reported as an MB₅₀ value; see present Example 3 and co-owned U.S. Patent Application Publication 2005/022683, herein incorporated by reference). As used herein, the term “MB₅₀” refers to the concentration of the binding peptide that gives a signal that is 50% of the maximum signal obtained in an ELISA-based binding assay. The MB₅₀ value provides an indication of the strength of the binding interaction or affinity of the components of the complex. The lower the value of the MB₅₀, the stronger the interaction of the peptide with its corresponding substrate. In one embodiment, the MB₅₀ value (reported in terms of molar concentration) for the hair-binding peptide is 10⁻⁴ M or less, preferably 10⁻⁶ M or less, more preferably 10⁻⁶ M or less, more preferably 10⁻⁷ M or less, and most preferably 10⁻⁸ M or less.

mRNA-Display

The present hair-binding peptides were biopanned against gray human hair using mRNA display, an in vitro method commonly used for identifying peptides having an affinity for a target material (U.S. Pat. No. 6,258,558). Briefly, a random library of DNA molecules is generated wherein they encode a peptide of a desired length. The length of the peptide within the display library is may be to be up to 200 amino acids in length and is typically designed to range from about 7 to about 100 amino acids in length. In one embodiment, the library of peptides is designed to be about 7 to about 60 amino acids in length, preferably about 7 to about 30 amino acids in length, more preferably about 15 to about 30 amino acids in length, and most preferably about 27 amino acids in length (i.e., a “27-mer” library). Typically, the nucleic acid molecule encoding the peptide includes (in addition to the coding region) appropriate 5′ and 3′ regulatory regions necessary for efficient in vitro transcription and translation. The design of the nucleic acid constructs used for preparing the mRNA-display library is well known to one of skill in the (see WO2005/051985). The nucleic acid molecules can be designed to optionally encode flexible linkers, cleavage sequences, fusion promoting sequences, and identification/purification tags (e.g., poly-A regions, His tags, etc.) to facility purification and/or processing in subsequence steps.

The library of random nucleic acid fragments is transcribed in vitro to produce an mRNA library. The mRNA is isolated and subsequently fused to a linker molecule (i.e., a puromycin-oligonucleotide linker or a puromycin derivative-oligonucleotide linker is used) using techniques well-known in the art (U.S. Pat. No. 6,258,558; U.S. Pat. No. 6,228,994; and Kurz et al., NAR, 28(18):e83 i-v (2000)). In a preferred embodiment, the puromycin-oligonucleotide linker comprises psoralen for rapid and facile preparation of the mRNA-protein fusions (Kurtz et al., supra). The mRNA-puromycin fusion molecules are then translated in vitro whereby the nascent polypeptide is fused via the puromycin-oligonucleotide linker to the mRNA (PROFUSION™ molecules; Adnexus Therapeutics, Weltham, Mass.). In this way, the phenotype (peptide) is linked to the corresponding genotype (RNA).

The mRNA-peptide fusion molecules are typically reverse transcribed into a DNA/mRNA-protein fusion molecules prior to affinity selection. The library, often comprising up to 10¹³ different sequences, is contacted with target ligand/material (typically an immobilized target and/or a solid surface). The selection process is carried out in an aqueous medium wherein parameters such as time, temperature, pH, buffer, salt concentration, and detergent concentration may be varied according the stringency of the selection strategy employed. Typically, the temperature of the incubation period ranges from 0° C. to about 40° C. and the incubation time ranges from about 1 to about 24 hours. The selection process is carried out in an aqueous medium wherein additional parameters such as pH, buffer, salt concentration, and detergent concentration may be varied according the stringency of the selection strategy employed.

Several washing steps are typically used to remove the non-binding/low affinity fusion molecules. The stringency of the washing conditions is adjusted to select those fusion molecules having the highest affinity for the target material (such as hair). The high affinity fusion molecules are isolated and then PCR-amplified in order to obtain the nucleic acid sequences encoding the hair-binding peptides. The mRNA-display selection cycle is typically repeated for 3 to 10 cycles in order to select/enrich those fusion molecules comprising peptide sequences exhibiting the highest affinity for the target material.

Error-prone PCR may optionally be incorporated into mRNA-display selection process whereby mutants derived from a previously selected high affinity sequence are used. The process is typically repeated for several cycles in order to obtain the peptides having improved affinity for the target material.

Optionally, any hair-binding peptide sequence identified using mRNA-display is verified using the free peptide. Typically, the nucleic acid molecule encoding the hair-binding peptide is cloned and recombinantly expressed in an appropriate microbial host cell, such as E. coli. The free peptide is then isolated and assayed against the targeted material to validate the binding affinity of the peptide sequence.

### Hair-Binding Peptides

Hair-binding peptides are short, single chain peptides that bind with high affinity to the surface of mammalian hair, preferably human hair. The present peptides were biopanned using mRNA-display against human gray hair. The hair-binding peptides can be used as a benefit agent or may be used to prepare peptide-based reagents for the delivery of a benefit agent to hair. As such, peptides having strong affinity for hair can be used to prepare peptide-based reagents capable coupling a benefit agent to the surface hair. In one embodiment, the benefit agent is a particulate benefit agent, such as a pigment.

The present hair-binding peptides are selected from the group consisting of SEQ ID NOs: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, and 54.

### Single Chain Peptide-Based Reagents for Coupling a Benefit Agent to Hair

The present hair-binding peptide may be used in hair care composition to couple a benefit agent to hair. The hair-binding peptide may also be used to form a protective layer on hair, and thus, may be considered as the benefit agent as well.

The hair-binding peptides can be coupled directly to the benefit agent or may be coupled to the benefit agent using a molecular spacer. As such, a peptide-based reagent is provided comprising the general structure:

(HBP)ₙ—BA

or

[(HBP)ₘ—S]ₙ—BA

wherein;
a) HBP is a hair-binding peptide;
b) BA is a benefit agent;
c) S is a molecular spacer;
d) m ranges from 1 to about 50; and
e) n ranges from 1 to about 1,000.

wherein the hair-binding peptide has a sequence selected from the group consisting of SEQ ID NOs: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, and 54.

It may also be desirable to have multiple hair-binding peptides coupled to the benefit agent (such as a coloring agent, a condition agent, a sunscreen agent or an antimicrobial agent) to enhance the interaction between the peptide-based reagent and the hair. Either multiple copies of the same hair-binding peptide or a combination of different hair-binding peptides may be used. In the case of large pigment particles, a large number of hair-binding peptides, i.e., up to about 1,000, may be coupled to the pigment. A smaller number of hair-binding peptides can be coupled to the smaller dye molecules, i.e., up to about 50. Therefore, in one embodiment of the present invention, the peptide-based hair colorants are diblock compositions comprise the structure above.

In another embodiment, the peptide-based hair colorants contain a spacer (S) separating the binding peptide from the hair coloring agent, as described above. Multiple copies of the hair-binding peptide may be coupled to a single spacer molecule. In this embodiment, the peptide-based hair colorants are triblock compositions consisting of at least one of the present hair-binding peptides, a spacer, and a coloring agent, having the general structure [(HBP)ₘ—S]ₙ-BA, where n ranges from 1 to about 1,000, preferably n is 1 to about 500, and m ranges from 1 to about 50, preferably m is 1 to about 10, and S is a spacer.

It should be understood that as used herein, HBP is a generic designation for the present hair-binding peptides. Where n or m as used above, is greater than 1, it is well within the scope of the invention to provide for the situation where a series of hair binding peptides of different sequences may form a part of the composition. Additionally, it should be understood that these structures do not necessarily represent a covalent bond between the peptide, the coloring agent, and the optional spacer. As described above, the coupling interaction between the hair-binding peptide, the coloring agent, and the optional spacer may be either covalent or non-covalent.

Single Chain Peptide-Based Reagents Comprising at least one Particulate Benefit Agent-Binding Peptide

The hair-binding peptide may be coupled to a particulate benefit agent using a benefit agent binding peptides. In one embodiment, the benefit agent-binding peptide is a pigment-binding peptide, a polymer-binding peptide, a clay-binding peptide, a calcium carbonate-binding peptide, or a silica-binding peptide.

In one embodiment, the single chain peptide-based reagent comprise a first one portion having affinity for hair (i.e., comprise at least one of the present hair-binding peptides) and a second portion having affinity for the surface of a particulate benefit agent (i.e., comprises at least one benefit agent-binding peptide). In one embodiment, the first portion comprises a plurality of hair-binding peptides with optional peptide linkers separating the various “finger” to form a hair-binding domain (“hand”). In another embodiment, the benefit agent-binding portion comprises a plurality of benefit agent-binding peptides optionally separated by one or more peptide spacers for form a benefit agent-binding “hand”. As such, a peptide-based reagent is also provided having the following structure:

a) a diblock peptide-based reagent having the general structure:

[(HBP)ₘ—(BABP)ₙ]ₓ; or

b) a triblock peptide-based reagent having the general structure:

[[(HBP)ₘ—S_{q}]ₓ−[(BABP)ₙ—Sᵣ]_{z}]_{y};

wherein;
i) HBP is a hair-binding peptide selected from the group consisting of SEQ ID NOs: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, and 54.
ii) BABP is a benefit agent-binding peptide having affinity for a hair benefit agent;
iii) S is a molecular spacer;
iv) m, n, x and z independently range from 1 to about 10;
v) y is from 1 to 5; and
vi) q an r are each independently 0 or 1, provided that both r and q may not be 0.

The molecular spacer is preferably a peptide spacer. Peptide-spacers used to link together two or more target surface-binding peptides will be referred to herein as “peptide linkers”. Peptide spacers that bridge together a hair binding hand to a benefit agent-binding hair will be referred to herein as “peptide bridges”.

The particulate benefit agent may comprise at least one applied coating. As such, the benefit agent-binding peptide may be selected to have strong affinity for the applied coating, such as a polymer, silica or any other cosmetically acceptable coating material. For optimal coupling of a particulate benefit agent to hair, the hair-binding hand should have stronger affinity for hair than the surface on the particulate benefit agent.

### Hair Care Benefit Agents

### Conditioning Agents

Hair conditioning agents (HCA) as herein defined are agents that improve the appearance, texture, and sheen of hair as well as increasing hair body or suppleness. Hair conditioning agents are well known in the art, see for example Green et al. (WO 01/07009) and are available commercially from various sources. Suitable examples of hair conditioning agents include, but are not limited to, cationic polymers, such as cationized guar gum, diallyl quaternary ammonium salt/acrylamide copolymers, quaternized polyvinylpyrrolidone and derivatives thereof, and various polyquaternium-compounds; cationic surfactants, such as stearalkonium chloride, centrimonium chloride, and Sapamin hydrochloride; fatty alcohols, such as behenyl alcohol; fatty amines, such as stearyl amine; waxes; esters; nonionic polymers, such as polyvinylpyrrolidone, polyvinyl alcohol, and polyethylene glycol; silicones; siloxanes, such as decamethylcyclopentasiloxane; polymer emulsions, such as amodimethicone; and nanoparticles, such as silica nanoparticles and polymer nanoparticles. The preferred hair conditioning agents of the present invention contain amine or hydroxyl functional groups to facilitate coupling to the hair-binding peptides, as described below. Examples of preferred conditioning agents are octylamine (CAS No. 111-86-4), stearyl amine (CAS No. 124-30-1), behenyl alcohol (CAS No. 661-19-8, Cognis Corp., Cincinnati, Ohio), vinyl group terminated siloxanes, vinyl group terminated silicone (CAS No. 68083-19-2), vinyl group terminated methyl vinyl siloxanes, vinyl group terminated methyl vinyl silicone (CAS No. 68951-99-5), hydroxyl terminated siloxanes, hydroxyl terminated silicone (CAS No. 80801-30-5), amino-modified silicone derivatives, [(aminoethyl)amino]propyl hydroxyl dimethyl siloxanes, [(aminoethyl)amino]propyl hydroxyl dimethyl silicones, and alpha-tridecyl-omega-hydroxy-poly(oxy-1,2-ethanediyl) (CAS No. 24938-91-8).

The peptide-based hair conditioners may be prepared by coupling a specific hair-binding peptide to a hair conditioning agent, either directly or via an optional spacer. The coupling interaction may be a covalent bond or a non-covalent interaction, such as hydrogen bonding, electrostatic interaction, hydrophobic interaction, or Van der Waals interaction. In the case of a non-covalent interaction, the peptide-based hair conditioner may be prepared by mixing the peptide with the conditioning agent and the optional spacer (if used) and allowing sufficient time for the interaction to occur. The unbound materials may be separated from the resulting peptide-based hair conditioner adduct using methods known in the art, for example, gel permeation chromatography.

The peptide-based hair conditioners may also be prepared by covalently attaching a specific hair-binding peptide to a hair conditioning agent, either directly or through a spacer. Any known peptide or protein conjugation chemistry may be used to form the peptide-based hair conditioners of the present invention. Conjugation chemistries are well-known in the art (see for example, G. T. Hermanson, supra). Suitable coupling agents include, but are not limited to, carbodiimide coupling agents, diacid chlorides, diisocyanates and other difunctional coupling reagents that are reactive toward terminal amine and/or carboxylic acid terminal groups on the peptides and to amine, carboxylic acid, or alcohol groups on the hair conditioning agent. The preferred coupling agents are carbodiimide coupling agents, such as 1-ethyl-3-(3-dimethylaminopropyl)-carbodiimide (EDC) and N,N′-dicyclohexyl-carbodiimide (DCC), which may be used to activate carboxylic acid groups for coupling to alcohol, and amine groups.

Additionally, it may be necessary to protect reactive amine or carboxylic acid groups on the peptide to produce the desired structure for the peptide-based hair conditioner. The use of protecting groups for amino acids, such as t-butyloxycarbonyl (t-Boc), are well known in the art (see for example Stewart et al., supra; Bodanszky, supra; and Pennington et al., supra). In some cases it may be necessary to introduce reactive groups, such as carboxylic acid, alcohol, amine, or aldehyde groups, on the hair conditioning agent for coupling to the hair-binding peptide. These modifications may be done using routine chemistry such as oxidation, reduction and the like, which is well known in the art.

It may also be desirable to couple the hair-binding peptide to the hair conditioning agent via a spacer. The spacer serves to separate the conditioning agent from the peptide to ensure that the agent does not interfere with the binding of the peptide to the hair. The spacer may be any of a variety of molecules, such as alkyl chains, phenyl compounds, ethylene glycol, amides, esters and the like. Preferred spacers are hydrophilic and have a chain length from 1 to about 100 atoms, more preferably, from 2 to about 30 atoms. Examples of preferred spacers include, but are not limited to ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, and ethyl, propyl, hexyl, steryl, cetyl, and palmitoyl alkyl chains. The spacer may be covalently attached to the peptide and the hair conditioning agent using any of the coupling chemistries described above. In order to facilitate incorporation of the spacer, a bifunctional cross-linking agent that contains a spacer and reactive groups at both ends for coupling to the peptide and the conditioning agent may be used. Suitable bifunctional cross-linking agents are well known in the art and include, but are not limited to diamines, such a as 1,6-diaminohexane; dialdehydes, such as glutaraldehyde; bis N-hydroxysuccinimide esters, such as ethylene glycol-bis(succinic acid N-hydroxysuccinimide ester), disuccinimidyl glutarate, disuccinimidyl suberate, and ethylene glycol-bis(succinimidylsuccinate); diisocyanates, such as hexamethylenediisocyanate; bis oxiranes, such as 1,4 butanediyl diglycidyl ether; dicarboxylic acids, such as succinyldisalicylate; and the like. Heterobifunctional cross-linking agents, which contain a different reactive group at each end, may also be used. Examples of heterobifunctional cross-linking agents include, but are not limited to compounds having the following structure:

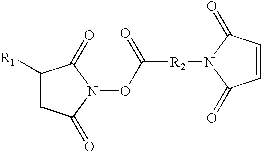

where: R₁ is H or a substituent group such as —SO₃Na, —NO₂, or —Br; and R₂ is a spacer such as —CH₂CH₂ (ethyl), —(CH₂)₃ (propyl), or —(CH₂)₃C₆H₅ (propyl phenyl). An example of such a heterobifunctional cross-linking agent is 3-maleimidopropionic acid N-hydroxysuccinimide ester. The N hydroxysuccinimide ester group of these reagents reacts with amine or alcohol groups on the conditioner, while the maleimide group reacts with thiol groups present on the peptide. A thiol group may be incorporated into the peptide by adding a cysteine group to at least one end of the binding peptide sequence (i.e., the C-terminus or N-terminus). Several spacer amino acid residues, such as glycine, may be incorporated between the binding peptide sequence and the terminal cysteine to separate the reacting thiol group from the binding sequence.

The spacer may be a peptide composed of any amino acid and mixtures thereof. The preferred peptide spacers are composed of the amino acids glycine, alanine, and serine, and mixtures thereof. In addition, the peptide spacer may contain a specific enzyme cleavage site, such as the protease Caspase 3 site, given by SEQ ID NO: 55, which allows for the enzymatic removal of the conditioning agent from the hair. The peptide spacer may be from 1 to about 60 amino acids, preferably from 3 to about 50 amino acids. These peptide spacers may be linked to the hair-binding peptide by any method known in the art. For example, the entire binding peptide-peptide spacer diblock may be prepared using the standard peptide synthesis methods described supra. In addition, the hair-binding peptide and peptide spacer blocks may be combined using carbodiimide coupling agents (G. T. Hermanson, supra), diacid chlorides, diisocyanates and other difunctional coupling reagents that are reactive to terminal amine and/or carboxylic acid terminal groups on the peptides. Alternatively, the entire hair binding peptide-peptide spacer diblock may be prepared using the recombinant DNA and molecular cloning techniques described herein. The spacer may also be a combination of a peptide spacer and an organic spacer molecule, which may be prepared using the methods described above.

It may also be desirable to have multiple hair-binding peptides coupled to the hair conditioning agent to enhance the interaction between the peptide-based hair conditioner and the hair. Either multiple copies of the same hair-binding peptide or a combination of different hair-binding peptides may be used. In the case of large conditioning particles (such as particle emulsions), a large number of hair-binding peptides, i.e. up to about 1,000, may be coupled to the conditioning agent. A smaller number of hair-binding peptides can be coupled to the smaller conditioner molecules, i.e., up to about 50. Therefore, in one embodiment, the peptide-based reagents consisting of a hair-binding peptide (HBP) and a benefit agent (BA), wherein the benefit agent is a hair-conditioning agent (HCA), having the general structure (HBP)ₙ—BA, where n ranges from 1 to about 1,000, preferably from 1 to about 50; wherein the hair-binding peptide is selected from the group consisting of SEQ ID NOs: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, and 54. In another embodiment, the peptide-based hair conditioners contain a spacer (S) separating the hair-binding peptide from the hair conditioning agent, as described above. Multiple copies of the hair-binding peptide may be coupled to a single spacer molecule. In this embodiment, the peptide-based hair conditioners are triblock compositions consisting of a hair-binding peptide, a spacer, and a benefit agent (BA) that is a hair conditioning agent (HCA), having the general structure [(HBP)ₘ—S]ₙ BA, where n ranges from 1 to about 1,000, preferably n is 1 to about 50, and m ranges from 1 to about 50, preferably m is 1 to about 10.

It should be understood that as used herein, HBP is a generic designation referring to any one of the present hair-binding peptides described herein. Where n or m as used above, is greater than 1, it is well within the scope of the invention to provide for the situation where a series of hair-binding peptides of different sequences may form a part of the composition. Additionally, it should be understood that these structures do not necessarily represent a covalent bond between the peptide, the hair conditioning agent, and the optional spacer. As described above, the coupling interaction between the peptide, the hair conditioning agent, and the optional spacer may be either covalent or non-covalent.

The peptide-based hair conditioners of the present invention may be used in compositions for hair care. It should also be recognized that the hair-binding peptides themselves can serve as conditioning agents for the treatment of hair. Hair care compositions are herein defined as compositions for the treatment of hair, including but not limited to shampoos, conditioners, lotions, aerosols, gels, mousses, and hair dyes comprising an effective amount of a peptide-based hair conditioner or a mixture of different peptide-based hair conditioners in a cosmetically acceptable medium. An effective amount of a peptide-based hair conditioner or hair-binding peptide for use in a hair care composition is herein defined as a proportion of from about 0.01% to about 10%, preferably about 0.01% to about 5% by weight relative to the total weight of the composition. Components of a cosmetically acceptable medium for hair care compositions are described by Philippe et al. in U.S. Pat. No. 6,280,747, and by Omura et al. in U.S. Pat. No. 6,139,851 and Cannell et al. in U.S. Pat. No. 6,013,250. For example, these hair care compositions can be aqueous, alcoholic or aqueous-alcoholic solutions, the alcohol preferably being ethanol or isopropanol, in a proportion of from about 1 to about 75% by weight relative to the total weight, for the aqueous-alcoholic solutions. Additionally, the hair care compositions may contain one or more conventional cosmetic or dermatological additives or adjuvants including but not limited to, antioxidants, preserving agents, fillers, surfactants, UVA and/or UVB sunscreens, fragrances, thickeners, wetting agents and anionic, nonionic or amphoteric polymers, and dyes or pigments.

### Peptide-Based Hair Colorants

The peptide-based hair colorants may be formed by coupling at least one of the present hair-binding peptides (HBP) with a benefit agent that acts as a coloring agent (C). The hair-binding peptide part of the peptide-based hair colorant binds strongly to the hair, thus keeping the coloring agent attached to the hair for a long lasting hair coloring effect. The hair-binding peptides are selected from the group consisting of SEQ ID NOs: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, and 54. Additionally, any known hair-binding peptide may be used in combination with one or more of the present hair-binding peptides including, but not limited to those described by Janssen et al. in U.S. Patent Application Publication No. 2003/0152976; Janssen et al. in WO 04048399; U.S. Pat. No. 7,220,405; and U.S. patent application Ser. Nos. 11/074,473; 11/359,163; and 11/251,715.

Coloring agents as herein defined are any dye, pigment, lake, and the like that may be used to change the color of hair. In the peptide-based hair colorants of the present invention, any known coloring agent may be used. Hair coloring agents are well known in the art (see for example Green et al. supra, CFTA International Color Handbook, 2nd ed., Micelle Press, England (1992) and Cosmetic Handbook, US Food and Drug Administration, FDA/IAS Booklet (1992)), and are available commercially from various sources (for example Bayer, Pittsburgh, Pa.; Ciba-Geigy, Tarrytown, N.Y.; ICI, Bridgewater, N.J.; Sandoz, Vienna, Austria; BASF, Mount Olive, N.J.; and Hoechst, Frankfurt, Germany). Suitable hair coloring agents include, but are not limited to dyes, such as 4-hydroxypropylamino-3-nitrophenol, 4-amino-3-nitrophenol, 2-amino-6-chloro-4-nitrophenol, 2-nitro-paraphenylenediamine, N,N-hydroxyethyl-2-nitro-phenylenediamine, 4-nitro-indole, Henna, HC Blue 1, HC Blue 2, HC Yellow 4, HC Red 3, HC Red 5, Disperse Violet 4, Disperse Black 9, HC Blue 7, HC Blue 12, HC Yellow 2, HC Yellow 6, HC Yellow 8, HC Yellow 12, HC Brown 2, D&C Yellow 1, D&C Yellow 3, D&C Blue 1, Disperse Blue 3, Disperse violet 1, eosin derivatives such as D&C Red No. 21 and halogenated fluorescein derivatives such as D&C Red No. 27, D&C Red Orange No. 5 in combination with D&C Red No. 21 and D&C Orange No. 10; and pigments, such as D&C Red No. 36 and D&C Orange No. 17, the calcium lakes of D&C Red Nos. 7, 11, 31 and 34, the barium lake of D&C Red No. 12, the strontium lake of D&C Red No. 13, the aluminum lakes of FD&C Yellow No. 5, of FD&C Yellow No. 6, of D&C Red No. 27, of D&C Red No. 21, and of FD&C Blue No. 1, iron oxides, manganese violet, chromium oxide, titanium dioxide, titanium dioxide nanoparticles, zinc oxide, barium oxide, ultramarine blue, bismuth citrate, and carbon black particles. The preferred hair coloring agents of the present invention are D&C Yellow 1 and 3, HC Yellow 6 and 8, D&C Blue 1, HC Blue 1, HC Brown 2, HC Red 5,2-nitro-paraphenylenediamine, N,N-hydroxyethyl-2-nitro-phenylenediamine, 4-nitro-indole, and carbon black. In one embodiment, the coloring agent is a pigment particle, a coated pigment particle, and mixtures thereof.

Metallic and semiconductor nanoparticles may also be used as hair coloring agents due to their strong emission of light (U.S. Patent Application Publication No. 2004/0010864 to Vic et al.). The metallic nanoparticles include, but are not limited to, particles of gold, silver, platinum, palladium, iridium, rhodium, osmium, iron, copper, cobalt, and alloys composed of these metals. An “alloy” is herein defined as a homogeneous mixture of two or more metals. The “semiconductor nanoparticles” include, but are not limited to, particles of cadmium selenide, cadmium sulfide, silver sulfide, cadmium sulfide, zinc oxide, zinc sulfide, zinc selenide, lead sulfide, gallium arsenide, silicon, tin oxide, iron oxide, and indium phosphide. The nanoparticles are stabilized and made water-soluble by the use of a suitable organic coating or monolayer. As used herein, monolayer-protected nanoparticles are one type of stabilized nanoparticle. Methods for the preparation of stabilized, water-soluble metal and semiconductor nanoparticles are known in the art, and are described by Huang et al. in U.S. patent application Ser. No. 10/622,889. The color of the nanoparticles depends on the size of the particles. Therefore, by controlling the size of the nanoparticles, different colors may be obtained. For example, ZnS-coated CdSe nanoparticles cover the entire visible spectrum over a particle size range of 2 to 6 nm. Specifically, CdSe nanoparticles with a core size of 2.3, 4.2, 4.8 and 5.5 nm emit light at the wavelength centered around 485, 565, 590, and 625 nm, respectively. Water-soluble nanoparticles of different sizes may be obtained from a broad size distribution of nanoparticles using the size fractionation method described by Huang, supra. That method comprises the regulated addition of a water-miscible organic solvent to a solution of nanoparticles in the presence of an electrolyte. Increasing additions of the water-miscible organic solvent result in the precipitation of nanoparticles of decreasing size. The metallic and semiconductor nanoparticles may also serve as volumizing agents, as described above.

Of particular utility are titanium dioxide nanoparticles that not only serve as a colorant but additionally may serve to block harmful UV radiation. Suitable titanium dioxide nanoparticles are described in U.S. Pat. NOs. 5,451,390; 5,672,330; and 5,762,914. Titanium dioxide P25 is an example of a suitable commercial product available from Degussa. Other commercial suppliers of titanium dioxide nanoparticles include Kemira, Sachtleben, and Tayca.

The titanium dioxide nanoparticles typically have an average particle size diameter of less than 100 nanometers (nm) as determined by dynamic light scattering which measures the particle size distribution of particles in liquid suspension. The particles are typically agglomerates which may range from about 3 nm to about 6000 nm. Any process known in the art can be used to prepare such particles. The process may involve vapor phase oxidation of titanium halides or solution precipitation from soluble titanium complexes, provided that titanium dioxide nanoparticles are produced.

A preferred process to prepare titanium dioxide nanoparticles is by injecting oxygen and titanium halide, preferably titanium tetrachloride, into a high-temperature reaction zone, typically ranging from 400 to 2000 degrees centigrade. Under the high temperature conditions present in the reaction zone, nanoparticles of titanium dioxide are formed having high surface area and a narrow size distribution. The energy source in the reactor may be any heating source such as a plasma torch.

Additionally, the coloring agent may be a colored, polymeric microsphere. Exemplary polymeric microspheres include, but are not limited to, microspheres of polystyrene, polymethylmethacrylate, polyvinyltoluene, styrene/butadiene copolymer, and latex. For use in the invention, the microspheres have a diameter of about 10 nanometers to about 2 microns. The microspheres may be colored by coupling any suitable dye, such as those described above, to the microspheres. The dyes may be coupled to the surface of the microsphere or adsorbed within the porous structure of a porous microsphere. Suitable microspheres, including undyed and dyed microspheres that are functionalized to enable covalent attachment, are available from companies such as Bang Laboratories (Fishers, Ind.).

The peptide-based hair colorants may be prepared by coupling at least one of the present hair-binding peptides to a coloring agent, either directly or via a spacer. Any of the coupling methods described above may be used. It may be necessary to introduce reactive groups, such as carboxylic acid, alcohol, amine, or aldehyde groups, on the coloring agent for coupling to the hair-binding peptide covalently. These modifications may be done using routine chemistry, which is well known in the art. For example, the surface of carbon black particles may be oxidized using nitric acid, a peroxide such as hydrogen peroxide, or an inorganic initiator such as ammonium persulfate, to generate functional groups. Preferably, the carbon black surface is oxidized using ammonium persulfate as described by Carrasco-Marin et al. (J. Chem. Soc., Faraday Trans. 93:2211-2215 (1997)). Amino functional groups may be introduced to the surface of carbon black using an organic initiator such as 2,2′-Azobis(2-methylpropionamide)-dihydrochloride. The inorganic pigments and the nanoparticles may be derivatized to introduce carboxylic acid or amino functional groups in a similar manner.

### Pigment-Binding Peptides

In one embodiment, the particulate benefit agent may be a pigment, a coated-pigment, or a mixture thereof. Peptide-based reagents may be prepared linking one or more of the present hair-binding peptides and one or more pigments. These bifunctional reagents may be used to delivery a pigment or coated pigment to the surface of hair (See U.S. Pat. No. 7,285,264). In one embodiment, the peptide based reagent comprise at least one hair-binding domain and at least one pigment-binding domains, wherein the domains linked together by an optional spacer.

Particulate benefit agents, including pigments, may be coated with one or more polymers known in the art. Polymer-binding peptides and/or polymer-binding domains may be linked to one or more of the present hair-binding peptides to form two-handed peptide reagents suitable for coupling a polymer coated benefit agent to the surface of hair (see U.S. Patent Application Publication No. 2007-0065387). Examples of peptides having affinity for various polymers have also been reported.

The pigment may also be coated with silica, a material that is often used as a coating on pigment particles. As such, silica-binding peptides may be used to in the peptide-based reagent to couple hair to a silica-coated particle.

A non-limiting list of benefit agent-binding peptides, including peptides that bind to particulates and particulate coating materials is provided including peptides that bind to materials such as poly(methyl methacrylate) (SEQ ID NOs. 56-82; U.S. Patent Application Publication No. 2007-0265431), polypropylene (SEQ ID NOs: 83-89; U.S. Patent Application Publication No. 2007-0264720), polyethylene (SEQ ID NOs. 99-105; U.S. Patent Application Publication No. 2007-0141628), poly tetrafluoroethylene (SEQ ID NOs. 90-98; U.S. patent application Ser. No. 11/607,734), nylon (SEQ ID NOs 106-111; U.S. Patent Application Publication No. 2007-0141629), polystyrene (SEQ ID NOs. 112-114; U.S. Patent Application Publication No. 2007-0261775), cellulose acetate (SEQ ID NOs. 115-118; U.S. Provisional Patent Application No. 61/016,708), carbon black (SEQ ID NOs. 119-122; U.S. Patent Application Publication No. 2005-0054752), CROMOPHTAL® yellow (SEQ ID NOs. 123-131; U.S. Patent Application Publication No. 2005-0054752; available from BASF Corp., Florham Park, N.J.), SUNFAST® magenta (SEQ ID NOs. 132-134; US 2005-0054752; Sun Chemical Co., Parsippany, N.J.), SUNFAST® blue (SEQ ID NOs. 135-143; U.S. Patent Application Publication No. 2005-0054752), silica (SEQ ID NOs. 215-237; co-filed, copending, and co-owned U.S. Provisional Patent Application No. 61/138,631), iron oxide (SEQ ID NOs. 144-173; co-filed, copending, and co-owned U.S. Provisional Patent Application No. 61/138,623), clay (SEQ ID NOs. 174-188; U.S. Patent Application Publication No. 2007-0249805), and calcium carbonate (SEQ ID NOs. 189-214; U.S. patent application Ser. No. 11/828,539).

### Hair Care Compositions

The benefit agent may include any compound or material that provides benefit to hair and typically includes, but is not limited to colorants, conditioners, sunscreen agents, antimicrobial agents, and the like. “Hair care compositions” are herein defined as compositions for the treatment of hair including, but not limited to, shampoos, conditioners, rinses, lotions, aerosols, gels, and mousses.

An “effective amount” of the peptide-based reagent and benefit agent (combined wt %) for use in hair care compositions is a concentration of about 0.001% to about 20%, preferably about 0.01% to about 10% by weight relative to the total weight of the composition. This proportion may vary as a function of the type of hair care composition.

The concentration of the peptide-based hair reagent in relation to the concentration of the benefit agent (e.g., a pigment) may need to be optimized for best results. Additionally, a mixture of different peptide-based hair reagents (such as peptide-based hair colorants) having an affinity for different pigments may be used in the composition. The peptide-based reagents in the mixture need to be chosen so that there is no interaction between the peptides that mitigates the beneficial effect. Suitable mixtures of peptide-based hair reagents may be determined by one skilled in the art using routine experimentation. If a mixture of peptide-based hair coloring reagents is used in the composition, the total concentration of the reagents is about 0.001% to about 20% by weight relative to the total weight of the composition.

The composition may further comprise a cosmetically acceptable medium for hair care compositions, examples of which are described by Philippe et al. in U.S. Pat. No. 6,280,747, and by Omura et al. in U.S. Pat. No. 6,139,851 and Cannell et al. in U.S. Pat. No. 6,013,250. For example, these hair care compositions can be aqueous, alcoholic or aqueous-alcoholic solutions, the alcohol preferably being ethanol or isopropanol, in a proportion of from about 1 to about 75% by weight relative to the total weight for the aqueous-alcoholic solutions. Additionally, the hair care compositions may contain one or more conventional cosmetic or dermatological additives or adjuvants including, but not limited to, antioxidants, preserving agents, fillers, surfactants, UVA and/or UVB sunscreens, fragrances, thickeners, wetting agents and anionic, nonionic or amphoteric polymers, and dyes.

### Production of Hair-Binding Peptides

Peptides may be prepared using standard peptide synthesis methods, which are well known in the art (see for example Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, peptides may be prepared using recombinant DNA and molecular cloning techniques. Genes encoding the peptides may be produced in heterologous host cells, particularly in the cells of microbial hosts.

Preferred heterologous host cells for expression of the hair-binding peptides of the present invention are microbial hosts that can be found broadly within the fungal or bacterial families and which grow over a wide range of temperature, pH values, and solvent tolerances. Because transcription, translation, and the protein biosynthetic apparatus are the same irrespective of the cellular feedstock, functional genes are expressed irrespective of carbon feedstock used to generate cellular biomass. Examples of host strains include, but are not limited to, fungal or yeast species such as Aspergillus, Trichoderma, Saccharomyces, Pichia, Candida, Yarrowia, Hansenula, or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium, and Klebsiella.

A variety of expression systems can be used to produce the peptides of the present invention. Such vectors include, but are not limited to, chromosomal, episomal and virus-derived vectors, e.g., vectors derived from bacterial plasmids, from bacteriophage, from transposons, from insertion elements, from yeast episomes, from viruses such as baculoviruses, retroviruses and vectors derived from combinations thereof such as those derived from plasmid and bacteriophage genetic elements, such as cosmids and phagemids. The expression system constructs may contain regulatory regions that regulate as well as engender expression. In general, any system or vector suitable to maintain, propagate or express polynucleotide or polypeptide in a host cell may be used for expression in this regard. Microbial expression systems and expression vectors contain regulatory sequences that direct high level expression of foreign proteins relative to the growth of the host cell. Regulatory sequences are well known to those skilled in the art and examples include, but are not limited to, those which cause the expression of a gene to be turned on or off in response to a chemical or physical stimulus, including the presence of regulatory elements in the vector, for example, enhancer sequences. Any of these could be used to construct chimeric genes for production of the any of the hair-binding peptides of the present invention. These chimeric genes could then be introduced into appropriate microorganisms via transformation to provide high level expression of the hair-binding peptides.

Vectors or cassettes useful for the transformation of suitable host cells are well known in the art. Typically the vector or cassette contains sequences directing transcription and translation of the relevant gene, one or more selectable markers, and sequences allowing autonomous replication or chromosomal integration. Suitable vectors comprise a region 5′ of the gene, which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. Selectable marker genes provide a phenotypic trait for selection of the transformed host cells such as tetracycline or ampicillin resistance in E. coli.

Initiation control regions or promoters which are useful to drive expression of the chimeric gene in the desired host cell are numerous and familiar to those skilled in the art. Virtually any promoter capable of driving the gene is suitable for producing the binding peptides of the present invention including, but not limited to: CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, araB, tet, trp, P_{L}, P_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

The vector containing the appropriate DNA sequence is typically employed to transform an appropriate host to permit the host to express the peptide of the present invention. Cell-free translation systems can also be employed to produce such peptides using RNAs derived from the DNA constructs of the present invention. Optionally it may be desired to produce the instant gene product as a secretion product of the transformed host. Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal which is functional in the production host. Methods for choosing appropriate signal sequences are well known in the art (see for example EP 546049 and WO 9324631). The secretion signal DNA or facilitator may be located between the expression-controlling DNA and the instant gene or gene fragment, and in the same reading frame with the latter.

### Methods for Treating Hair

Methods are provided for treating hair with a benefit agent such as peptide-based conditioners, colorants, sunscreen agents, and antimicrobial agents of the present invention. In another embodiment, a method is provided for forming a protective film of peptide-based conditioner on hair by applying one of the compositions described above comprising an effective amount of a peptide-based hair conditioner to the hair and allowing the formation of the protective film. The compositions may be applied to hair by various means, including, but not limited to spraying, brushing, and applying by hand. The peptide-based conditioner composition is left in contact with hair for a period of time sufficient to form the protective film, preferably for at least about 0.1 min to 60 min.

In one embodiment, a method to form a protect layer on hair is provided comprising:
a) providing a hair-care composition comprising at least one peptide-based reagent comprising at least one of the present hair-binding peptides; and
b) contacting hair with the hair-care composition of (a) whereby the peptide-based reagent adheres to hair.

A method is also provided for coloring hair by applying a hair care composition comprising an effective amount of a peptide-based reagent and the benefit agent to the hair by means described above. The hair care composition is allowed to contact the hair for a period of time sufficient to cause the desired effect (coloration, conditioning, forming a protective layer, etc.) to the hair, preferably between about 5 seconds to about 50 minutes, and more preferably from about 5 seconds to about 60 seconds, and then the hair care composition may be rinsed from the hair.

### EXAMPLES

The present invention is further defined in the following Examples.

It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “sec” means second(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmole” means micromole(s), “g” means gram(s), “μg”means microgram(s), “mg” means milligram(s), “g” means the gravitation constant, “rpm” means revolution(s) per minute, “pfu” means plaque forming unit(s), “BSA” means bovine serum albumin, “ELISA” means enzyme linked immunosorbent assay, “IPTG” means isopropyl β-D-thiogalactopyranoside, “A” means absorbance, “A₄₅₀” means the absorbance measured at a wavelength of 450 nm, “TBS” means Tris-buffered saline, “TBST” means Tris-buffered saline containing TWEEN® 20, “TMB” means 3,3′,5,5′-tetramethylbenzidine, “DEPC” means diethylpyrocarbonate, and “HRP” means horse radish peroxidase.

### General Methods:

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

Materials and Methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or by Thomas D. Brock in Biotechnology: A Textbook of Industrial Microbiology, Second Edition, Sinauer Associates, Inc., Sunderland, Mass., 1989. All reagents, restriction enzymes and materials used for the growth and maintenance of bacterial cells were obtained from BD Diagnostic Systems (Sparks, Md.), Invitrogen (Carlsbad, Calif.), Life Technologies (Rockville, Md.), QIAGEN (Valencia, Calif.), Sigma-Aldrich Chemical Company (St. Louis, Mo.) or Pierce Chemical Co. (A division of Thermo Fisher Scientific Inc., Rockford, Ill.) unless otherwise specified. unless otherwise specified.

### Example 1

### Selection of Hair Binding Peptides Using mRNA-Display Biopanning

The purpose of this Example was to demonstrate enrichment and isolation of hair-binding peptides using an mRNA display biopanning method.

mRNA-Display Peptide Libraries:

Methods to make libraries of DNA molecules suitable as starting materials for mRNA-display are well-known in the art (see WO2005/051985). The following procedure was used to identify 27-mer peptides that have a specific affinity for hair as target material.

Briefly, a library of random nucleic acid molecules (dsDNA) each molecule encoding a peptide of desired length was generated. A linear peptide library containing 81 nucleotide positions or 27 randomized amino acid positions was used (“p27 library”). The p27 library was designed to include appropriate 5′ and 3′ regions for efficient in vitro transcription, translation, purification, and coupling to the MHA-oligonucleotide linker (MHA is 3′-[α-amino-p-methoxy-hydrocinnamido]-3′-deoxy-adenosine) in the individual molecules.

The DNA encoding the linear peptide library was designed to include a T7 promoter and a tobacco mosaic virus (TMV) translation initiation sequence operably linked to the coding sequence (CDS) (Liu et al., Methods in Enzymology, 318:268-293 (2000)). The CDS was designed to encode: (1) a constant N-terminal flaking region comprising a hexa-histidine tag followed by a flexible linker (underlined) sequence (MHHHHHHSGSSSGSGSG; SEQ ID NO: 1), (2) the randomized 27-mer linear peptide, and (3) a constant C-terminal flanking region (TSGGSSGSSLGVASAI; SEQ ID NO: 2) comprising another flexible linker region (bold) and a C-terminal sequence optimized for efficient coupling to the MHA-oligonucleotide linker (double-underlined).

### In Vitro Transcription

Double stranded DNA as result of the PCR reactions were transcribed into RNA using the RiboMax Express in vitro transcription kit (Promega Madison, Wis.). After incubation for at least 45 min at 37° C., DNase I was added and the incubation continued at 37° C. for additional 30 minutes to degrade all template DNA. The reaction mixture was purified by phenol/chloroform extraction. Then free nucleotides were removed by gel filtration using G25 microspin columns (Pharmacia; Milwaukee, Wis.). Concentration of purified RNA was determined by photometry at 260 nm.

### Library Preparation

Approximately 10 pmol of highly purified RNA was produced by in vitro transcription from the p27 DNA library and purified after DNase I digestion (by phenol/chloroform extraction and gel filtration, methods described below). The 3′-end of the p27 library RNA was modified by attachment of a MHA-linker molecule (as described ahead) and translated in vitro by means of a rabbit reticulocyte lysate. Covalent fusion products between peptide and coding RNA were purified on magnetic oligo(dT) beads, reverse transcribed, and again purified on a Ni-NTA purification matrix to remove uncoupled RNA and free peptides. About 8 μmol of peptide-RNA-cDNA-fusions were used as input for the first contact with target material during selection round 1.

### Chemical Coupling of RNA and MHA-Oligonucleotide Linker

Purified RNA was annealed (by heat denaturation for 1 minute at 85° C. and cooling down to 25° C. for 10 minutes) with a 1.5-fold excess of MHA-oligonucleotide linker-PEG₂A18 (5′-psoralen-UAG CGG AUG C A₁₈ (PEG-9)₂ CC-MHA [nucleotides shown in italics represent 2′-O-methyl-derivatives] (SEQ ID NO: 3). The covalent coupling was induced by radiation with UV-light (365 nm) for 15 min at room temperature. Aliquots of this reaction mixture before and after irradiation with UV were analyzed on a 6%-TBE-Urea-polyacrylamidgel to control the coupling efficiency (usually at least 60%).

### In Vitro Translation and ³⁵S-Labelling of Peptide-RNA Fusions

Ligated RNA was translated using a rabbit reticulocyte lysate from Promega in presence of 15 μCi ³⁵S-methionine (1000 Ci/mmole). After a 30 min incubation at 30° C., KCl and MgCl₂ were added to a final concentration of 530 mM and 150 mM respectively in order to promote formation of mRNA-peptide-fusions.

### Oligo(dT) Purification

For the purification of peptide-RNA-fusions from translation mixtures molecules were hybridized to magnetic oligo(dT) beads (Miltenyi Biotec; Bergisch Gladbach, Germany) in annealing buffer (100 mM Tris-HCl pH 8.0, 10 mM EDTA, 1 M NaCl and 0.25% Triton X-100) for 5 min at 4° C. Beads were separated from the mixture using MiniMACS-filtration columns (Miltenyi Biotec), repetitively washed with 100 mM Tris-HCl pH 8.0, 1 M NaCl, 0.25% Triton X-100 and finally eluted with water. A sample of this reaction was analyzed on 4-20% Tris/glycine-SDS-PAGE; radioactive bands were visualized using a PhosphoroImager.

### Reverse Transcription (RT)

The RNAs of Oligo(dT)-purified peptide-RNA-fusions were reverse transcribed using SuperScript II Reverse Transcriptase (Invitrogen, Carlsbad, Calif.) according to the manufacturers recommendations. RT reactions contained about 1.5-fold excess of 3′-ReversePrimer. A sample of this reaction was analyzed on 4-20% Tris/glycine-SDS-PAGE; radioactive bands were visualized using a PhosphorImager.

### His-taq Purification Purification by Ni²⁺-MAC (Metal Affinity Chromatography)

Reverse transcribed mRNA-peptide-fusion molecules were mixed with Ni-NTA-agarose (QIAGEN; Valencia, Calif.) in HBS buffer (20 mM HEPES pH 7.0, 150 mM NaCl, 0.025% Triton X-100, 100 μg/mL sheared salmon sperm DNA, 1 mg/mL BSA) and incubated for 60 min at room temperature under gentle shaking. Ni-NTA was then filtrated and washed with HNT buffer (20 mM HEPES pH 7.0, 150 mM NaCl, 0.025% Triton X-100) containing 5 mM imidazole. Finally peptide-RNA-cDNA-fusions were eluted with 150 mM imidazole in HNT buffer (20 mM HEPES pH 7.0, 150 mM NaCl, 0.025% Triton X-100). A sample of this reaction was analyzed on 4-20% Tris/glycine-SDS-PAGE; radioactive bands were visualized using a PhosphorImager. BSA (final concentration 1 mg/mL) and shared salmon sperm DNA (final concentration 100 μg/mL) were added to the eluates before contacting with target materials during selection step.

### Selection by Binding to Target Materials and Washing

A. Incubation of Peptide-RNA-cDNA-Fusion Library with Target Material:
Two Different Incubation Buffers were used:

1. HNTriton Incubation Buffer

Purified peptide-RNA-cDNA-fusions (PROFUSION™ molecules; Adnexus Therapeutics, Waltham, Mass.) after Ni-NTA purification were incubated for 60 minutes at room temperature in 1 mL (final volume) of 20 mM HEPES, pH 7.4, 150 mM NaCl, 1 mg/mL BSA, 100 μg/mL shared Salmon sperm DNA, 0.025% TritonX-100 in presence of DEPC-treated, blocked target material. Input activity of purified peptide-RNA-cDNA-fusions was determined by scintillation measurement.

2. HNTween Incubation Buffer

For additional stringency, purified peptide-RNA-cDNA-fusions were incubated for 60 minutes at room temperature in 1 mL (final volume) in HNTween buffer (20 mM Hepes, pH 7.4, 150 mM NaCl, 0.5% TWEEN® 20) with 1 mg/mL BSA, 100 μg/mL shared Salmon sperm DNA, DEPC-treated, blocked target material. Input activity of purified peptide-RNA-cDNA-fusions was determined by scintillation measurement.

### B. Washing:

Non-binding variants were washed away by one of the following washing procedures listed below:
Washing procedure A: used for washing the hair during selection round 1:

| | |
|---|---|
| 5x 5 sec. | each with HNTriton buffer (20 mM Hepes, pH 7.4, |
| | 150 mM NaCl, 0.025% Triton-X100) |
| 1x 5 sec | 150 mM NaCl (for buffer removal before elution |
| | with KOH) |

Washing procedure B: used for washing of hairs during selection round 2-9, and 13-control for gray hair:

| | |
|---|---|
| 1x 5 sec. | each with HNTween buffer (20 mM Hepes, pH 7.4, |
| | 150 mM NaCl, 0.5% Tween-20) |
| 2x 5 min. | with 10% shampoo in HNTriton buffer |
| 2x 5 sec | with HNTween buffer |
| 1x 5 sec | 150 mM NaCl (for buffer removal before elution |
| | with KOH) |

Washing procedure C: used for washing of hair during selection rounds 10, 12, and 13 for gray hair.

| | |
|---|---|
| 2x 5 sec. | each with HNTween buffer (20 mM Hepes, pH 7.4, |
| | 150 mM NaCl, 0.5% Tween-20) |
| 1x 5 min. | with 10% shampoo in HNTriton buffer |
| 1x 5 sec | with HNTween buffer including tube change |
| 4x 30 min | with 10% shampoo in HNTriton buffer |
| 3x 5 sec | with HNTween buffer; 1 tube change during the |
| | third wash |
| 1x 5 sec | 150 mM NaCl (for buffer removal before elution |
| | with KOH) |

Washing procedure D used for washing of grey hair during selection round 11:

| | |
|---|---|
| 2x 5 sec. | each with HNTween buffer (20 mM Hepes, pH 7.4, |
| | 150 mM NaCl, 0.5% Tween-20) |
| 1x 5 min. | with 10% Shampoo in HNTriton buffer |
| 1x 5 sec | with HNTween buffer including tube change |
| 2x 30 min | with 10% shampoo in HNTriton buffer |
| 1x 60 min | with 10% shampoo in HNTriton buffer in presence |
| | of 10 fresh grey hairs (to prevent rebinding |
| | effects) |
| 3x 5 sec | with HNTween buffer; 1 tube change during the |
| | third wash |
| 1x 5 sec | 150 mM NaCl (for buffer removal before elution |
| | with KOH) |

The shampoo used in the above washing procedures was a commercially available hair shampoo having the following composition:

| | |
|---|---|
| Water | 51% |
| Ammonium lauryl sulfate | 20% |
| Sodium lauryl ether sulfate | 15% |
| Cocamidopropyl betaine | 7% |
| Cocamide MEA | 2.5% |
| Miscellaneous minor components** | ~4.5% |

**(e.g. various pH adjusters, preservatives, vitamins, chelating agents, dispersants, lubricants, fragrances, and dyes)

### Comment on Incubation and Washing Conditions:

Normally during mRNA display selections a low detergent concentration is chosen to have low stringent conditions during up to 6 rounds of selection by keeping the detergent concentration at 0.025% Triton-X100. However, a higher stringency for the target material was applied from the beginning during incubation and washing (see washing procedures). The applied high concentrations of TWEEN®-20 and shampoo are close to the so called “critical micelle concentration” (CMC) allowing the formation of small micelles which might contain more than one peptide-RNA-cDNA-fusion. Since CMC driven aggregation of peptide-RNA-cDNA-fusions are critical for successful selections, higher concentrations of the detergents described above were not used.

cDNA Elution:

cDNAs of binding variants were eluted by incubation of target material in 50 μL of 100 mM KOH at 60° C. for 30 minutes. After centrifugation, supernatant was removed from target material and transferred into a fresh tube. KOH eluates were subsequently neutralized by addition of 1 μL of 1 M Tris/HCl, pH 7.0 and 3.8 μL of 1 M HCl (per 50 μL 100 mM KOH).

### Polymerase Chain Reaction (PCR):

After elution in KOH and neutralization, the recovered cDNAs were amplified by quantitative PCR with increasing numbers of amplification cycles (12, 15, 18, 21, 24 and 27 cycles). Products were subsequently analyzed by agarose gel electrophoresis over 2% agarose gels. Optimized conditions (minimal cycle number to get good enrichment of DNA of correct length) were then applied for a preparative PCR reaction and controlled again by agarose gel electrophoresis.

Analytical and preparative PCR reactions were performed in presence of 10 mM Tris-HCl (pH 8.8 at 25° C.), 50 mM KCl, 0.08% Nonidet P40, 2 mM MgCl₂, 2.5 mM dNTPs, 1 μM of each forward and reverse primer (5′-TAATACGACTCATAGGGACAATTACTATTTACAA TTACAATG-3′; SEQ ID NO: 4) and (5′-AATTAAATAGCGGATGCTACACCAAGACTAGAACCGCTG-3′; SEQ ID NO: 5), ⅕ volume of neutralized cDNA eluate and 0.05 U/μL Tag polymerase (Promega). Temperature program of PCR reaction is given below: Initial denaturation: 90 sec at 94° C.; cycling: 15 sec at 94° C. (denaturation), 20 sec at 60° C. (annealing), 30 sec at 72° C. (extension); post treatment: 3 min at 72° C. (post-treatment); hold at 4° C.

Enrichment of cDNA-RNA-Peptide Fusion Molecules Binding to Gray Hair

Initial selection was conducted using stringent washing conditions. Thirteen rounds of selection were conducted and the relative binding of radioactively labeled cDNA-RNA-peptide fusion molecules to the gray hair target material was measured. The amount of target used per round was two hair twirled into a hairpin-like structure.

Rounds 1 of selection used washing procedure A. Rounds 2-9 and 13-control used washing procedure B. Rounds 10, 12, and 13 used washing procedure C. Round 11 used washing procedure D. The relative amount of enrichment (reported as percent enrichment of binding molecules relative to their respective input signals [activity of cDNA-RNA-peptide fusions before contacting with the target material]) is provided in Table 1.

TABLE 1

| | | | % Enrichment of cDNA- |
|---|---|---|---|
| | | | RNA-peptide fusion |
| Selection | Incubation | Washing | molecules having an |
| Round | Buffer | Procedure | affinity for gray hair |
| 1 | 1 | A | 0.0 |
| 2 | 1 | B | 0.307 |
| 3 | 1 | B | 1.289 |
| 4 | 1 | B | 1.629 |
| 5 | 1 | B | 2.502 |
| 6 | 1 | B | 0.996 |
| 7^{a} | 1 | B | 3.107^{a} |
| 8 | 2 | B | 1.591 |
| 9 | 2 | B | 2.155 |
| 10 | 2 | C | 0.736 |
| 11 | 2 | D | 1.291 |
| 12 | 2 | C | 0.546 |
| 13^{a} | 2 | C | 0.738^{a} |
| 13-control | 2 | B | 4.564 |

^{a}= enriched binder sequences of respective round analyzed

### Sequencing of 27-mer Gray Hair-Binding Peptides

The cDNA molecules from the enriched pool of gray hair-binding fusion molecules were isolated and PCR amplified as described above. The sequences of the DNA molecules encoding the gray hair-binding peptides were determined and are provided in Tables 2 (Round 7), and Table 3 (Round 13).

TABLE 2
Enriched Gray Hair-binding Peptides After
Round 7.

| | | SEQ ID |
|---|---|---|
| Sample No. | Amino Acid Sequence | NO: |
| 1 | TRSHVRIAMGYVWCSVKKFVSNLVDGS | 6 |
| 2 | RLRVSLVKKVLRTSTRQLWELLCSRLW | 7 |
| 3 | FKRRAKVWLACAKSRALLDYRKGFSWL | 8 |
| 4 | YKLFRFLVCKSEFFRGVSARFRGPTTT | 9 |
| 5 | THVVRTVKGLVCVSRIVSIWGRTWKAR | 10 |
| 6 | QKLCHKEKVSWDLRLMLHSVKRTLVAL | 11 |
| 7 | RVRKAFRGILVTFQDLCLDWRSLFRGT | 12 |
| 8, 12 | QLRRAKQLTNLWWHQSTKMWCSLARWT | 13 |
| 9 | LGSRLRGAYVKGLVWTKRLWLNAFACG | 14 |
| 10 | GGHYFTKIKQIALRIGVRMIRGYLYHW | 15 |
| 11 | GARQSRVRTLAIKVWCAVGVLSKKWNK | 16 |
| 13 | RLRKWSLGRSVVRAFTCRKNMPTFSSW | 17 |
| 14 | WRECRLKVAAKLRQKTLSTGNQIWIML | 18 |
| 15 | MGHVARRWMLHTVTTTLRGLSMCLGMV | 19 |
| 16 | FLARKLGHKGVVQVVGLDPAPHAAVRL | 20 |
| 17 | ATSPMKRLRRASRLVKMLPRLVSWLWM | 21 |
| 18 | HTRRQIVRPRDVFLMWCRLAKGSVKLW | 22 |
| 19 | GKRKWLCLRSTREMVGKAIGDLGSILL | 23 |
| 20 | ERLRVGRLQLVKGVKKIMRHLICL | 24 |
| 21 | RVKPGKRLKTRIITCMWDSVKGPWWT | 25 |
| 22 | SWKVWHRNRTGGP | 26 |
| 23 | WSRWCRTNKAASMLRAWKRLGRHWEVN | 27 |
| 24 | WRVRRLHRIVTGMCVSQYMRGSWLWT | 28 |
| 25 | SWKVWHRNKANRVH | 29 |
| 26 | GLLRRSAKSIISMTCKRPYLFSQLAKI | 30 |
| 27 | HTRRQIVRPRDVFLMWCRLAKGSVKLW | 31 |
| 28 | RRPVSFRVNKCRVRELASHGMVLWTYY | 32 |
| 29 | LLRRAKRSALALGKVMGQYGQMWCWG | 33 |
| 30 | FRQKCTRIRVRRQIWTYSTVGIRTWLA | 34 |

TABLE 3
Enriched Gray Hair-binding Peptides After
Round 13.

| | | SEQ |
|---|---|---|
| | | ID |
| Sample No. | Amino Acid Sequence | NO: |
| 1 | LRIKRVWLRVSGQVKCWFGWT | 35 |
| 2 | VRRRGHTTRIQAYIKLARFVANGVWQL | 36 |
| 3 | Not determined | — |
| 4, 6, 9, 29 | PWRRRIVWRFMRNHALASMLWLSVRTV | 37 |
| 5, 21, 24, | PLWRRITKRKLVRPVATLMWYWFTSKR | 38 |
| 26, 35 | | |
| 7 | Not determined | — |
| 8 | INRALRKLGSVLRCKSKARNTLGWAAW | 39 |
| 10, 14, 22, | PWRRRIVWRFMRNHALASMLWLSVSTV | 40 |
| 30, 39 | | |
| 11 | ERYRLFISRLVRPILHRAWCKLTRRKY | 41 |
| 12 | RSLRNLRWSLTRRRMTALGLYTMWALL | 42 |
| 13 | EFGFNTTHYRDNYYLQLQCIIRSRLVRSS | 43 |
| 15 | TRIRLLRRTKIVVRHGYKKLQCV | 44 |
| 16 | FGFNTTHYRDNYYLQLQCIIRSRLVRSS | 45 |
| 17 | Not determined | — |
| 18, 20, 37 | RLRSSKTLRHVLGKALDRTKQVWCLVT | 46 |
| 19 | RLRSSKTLQHVLGKALDRTKQVWCLVT | 47 |
| 23, 31, 38 | SRIKRVILKKGLVHKIRCLVKTQFSHL | 48 |
| 25, 27 | RLRFSTTLCRAVHHLKRMAVKVQIVSK | 49 |
| 28 | AVHVFRKLRRSVLWTRKVLLCTWLLR | 50 |
| 32 | LKKRLSAILHSLACIKPVKWKWGHRKI | 51 |
| 33 | Not determined | — |
| 34 | WWKKTLSEAWCTVKKDIAGLRKVIHGH | 52 |
| 36 | SRIKRVILKKGLVHKIRCLVETQFSHL | 53 |
| 40 | WFVHRSHKSRKSMVRSLWCWV | 54 |

### Example 2

### Determination of Hair-binding Affinity

The purpose of this Example was to determine the affinity of at least one hair-binding peptide for hair surfaces, measured as MB₅₀ values, using an ELISA assay to confirm that selection process produced hair-binding peptides with strong affinity for hair.

The peptide was synthesized using a standard solid phage synthesis method and was biotinylated by adding a biotinylated lysine residue at the C-terminus of the amino acid binding sequence for detection purposes. The peptide tested was CXH-G2 (see Sample ID Nos. 5, 21, 24, 26, 35 from Table 3; SEQ ID NO: 38)

The MB₅₀ measurement of the biotinylated peptide binding to hair was done using a gray hair bundle (90% gray human hairs were obtained from International Hair Importers and Products (Bellerose, N.Y.). The hair samples were assembled in bundles consisting of 100 hairs about 1 cm long which were bundled together using narrow tape at one end. The hair bundles were incubated in SUPERBLOCK® blocking buffer (Pierce Chemical) for 1 hour at room temperature (˜22° C.), followed by 3 washes with TBST (TBS in 0.05% TWEEN® 20). Peptide binding buffer consisting of various concentrations of biotinylated peptide in TBST and 1 mg/mL BSA was added to the hair bundles and incubated for 1 hour at room temperature, followed by 6 TBST washes. Then, the streptavidin-horseradish peroxidase (HRP) conjugate (Pierce Chemical Co., Rockford, Ill.) was added to each well (1.0 μg per well), and incubated for 1 h at room temperature, followed by 6 times of washes with TBST. All hair bundles were transferred to new tubes and then the color development (3,3′,5,5′-tetramethylbenzidine (TMB) was the substrate) and the absorbance measurements were performed following the manufacturer's protocol. The results were plotted as A₄₅₀ versus the concentration of peptide using GraphPad Prism 4.0 (GraphPad Software, Inc., San Diego, Calif.). The MB₅₀ value was calculated from Scatchard plots and is shown Table 4.

TABLE 4
MB₅₀ Value for Hair-Binding Peptide CXH-G2

| Peptide | Peptide Sequence | |
|---|---|---|
| ID NO. | (SEQ ID NO:) | MB₅₀ (M) |
| CXH-G2 | PLWRRITKRKLVRPVATLMWYWFTSKR- | 7.2 × 10⁻⁸ |
| | K(biotin)-NH₂ | |
| | (SEQ ID NO: 271) | |

What is claimed is:

1. A hair-binding peptide selected from the group consisting of SEQ ID NOs: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, and 54.

2. A peptide-based hair care reagent having the general structure:
(HBP)ₙ—BA
or
[(HBP)ₘ—S]ₙ—BA
wherein;
a) HBP is a hair-binding peptide;
b) BA is a benefit agent;
c) S is a molecular spacer;
d) m ranges from 1 to about 50; and
e) n ranges from 1 to about 1,000.
wherein the hair-binding peptide has a sequence selected from the group consisting of SEQ ID NOs: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, and 54.

3. The peptide-based reagent of claim 2 wherein the benefit agent is selected from the group consisting of colorants, conditioning agents, and antimicrobial agents.

4. The peptide-based reagent of claim 3 wherein the colorant is a dye, a lake, or a pigment.

5. The peptide-based hair care reagent of claim 3 wherein the conditioning agent is selected from the group consisting of cationic polymers, cationic surfactants; fatty alcohols, fatty amines, nonionic polymers, silicones, siloxanes, polymer emulsions, and nanoparticles.

6. The peptide-based reagent of claim 2 wherein the hair-binding peptide is generated by mRNA display.

7. A peptide-based reagent having the general structure:
a) a diblock peptide-based reagent having the general structure:
[(HBP)ₘ—(BABP)ₙ]ₓ; or
b) a triblock peptide-based reagent having the general structure:
[[(HBP)ₘ—S_{q}]ₓ-[(BABP)ₙ—Sᵣ]_{z}]_{y},;
wherein;
i) HBP is a hair-binding peptide selected from the group consisting of SEQ ID NOs: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, and 54.
ii) BABP is a benefit agent-binding peptide having affinity for a hair benefit agent;
iii) S is a molecular spacer;
iv) m, n, x and z independently range from 1 to about 10;
v) y is from 1 to 5; and
vi) q an r are each independently 0 or 1, provided that both r and q may not be 0.

8. The peptide-based reagent of claim 7 wherein the benefit agent-binding peptide is selected from the group consisting of pigment-binding peptides, polymer-binding peptides, clay-binding peptides, calcium carbonate-binding peptides, and silica-binding peptides

9. The peptide-based reagent of claim 7 wherein the hair-binding peptide is generated by mRNA display.

10. The peptide-based reagent of claim 7 wherein benefit agent is a particulate benefit agent.

11. The peptide-based reagent of claim 10 wherein the benefit agent is selected from the group consisting of pigments, conditioners, and sunscreen agents.

12. A hair-care composition comprising the hair-binding peptide of claim 1.

13. A hair-care composition comprising the peptide-based reagent of claim 2 or claim 7.

14. A method for applying a benefit agent, colorant, or conditioner to hair comprising:
a) providing the hair-care composition of claim 13; and
b) contacting hair with the hair care composition of (a) whereby the peptide-based reagent couples the benefit agent to hair.

15. A method for form a protective layer on hair is provided comprising:
a) providing the hair-care composition of claim 13; and
b) contact hair with the hair-care composition of (a) whereby the peptide-based reagent adheres to hair.


SEQUENCE LISTING (271 sequences)

SEQ ID NO: 1 - protein, 17 aa, artificial sequence
  N terminal constant region
MHHHHHHSGSSSGSGSG

SEQ ID NO: 2 - protein, 16 aa, artificial sequence
  C terminal constant region
TSGGSSGSSLGVASAI

SEQ ID NO: 3 - RNA, 28 nt, artificial sequence
  Synthetic oligonucleotide linker
uagcggaugcaaaaaaaaaaaaaaaaaa

SEQ ID NO: 4 - DNA, 42 bp, artificial sequence
  primer
taatacgactcatagggacaattactatttacaattacaatg

SEQ ID NO: 5 - DNA, 39 bp, artificial sequence
  primer
aattaaatagcggatgctacaccaagactagaaccgctg

SEQ ID NO: 6 - protein, 27 aa, artificial sequence
  hair-binding peptide
TRSHVRIAMGYVWCSVKKFVSNLVDGS

SEQ ID NO: 7 - protein, 27 aa, artificial sequence
  hair-binding peptide
RLRVSLVKKVLRTSTRQLWELLCSRLW

SEQ ID NO: 8 - protein, 27 aa, artificial sequence
  hair-binding peptide
FKRRAKVWLACAKSRALLDYRKGFSWL

SEQ ID NO: 9 - protein, 27 aa, artificial sequence
  hair-binding peptide
YKLFRFLVCKSEFFRGVSARFRGPTTT

SEQ ID NO: 10 - protein, 27 aa, artificial sequence
  hair-binding peptide
THVVRTVKGLVCVSRIVSIWGRTWKAR

SEQ ID NO: 11 - protein, 27 aa, artificial sequence
  hair-binding peptide
QKLCHKEKVSWDLRLMLHSVKRTLVAL

SEQ ID NO: 12 - protein, 27 aa, artificial sequence
  hair-binding peptide
RVRKAFRGILVTFQDLCLDWRSLFRGT

SEQ ID NO: 13 - protein, 27 aa, artificial sequence
  hair-binding peptide
QLRRAKQLTNLWWHQSTKMWCSLARWT

SEQ ID NO: 14 - protein, 27 aa, artificial sequence
  hair-binding peptide
LGSRLRGAYVKGLVWTKRLWLNAFACG

SEQ ID NO: 15 - protein, 27 aa, artificial sequence
  hair-binding peptide
GGHYFTKIKQIALRIGVRMIRGYLYHW

SEQ ID NO: 16 - protein, 27 aa, artificial sequence
  hair-binding peptide
GARQSRVRTLAIKVWCAVGVLSKKWNK

SEQ ID NO: 17 - protein, 27 aa, artificial sequence
  hair-binding peptide
RLRKWSLGRSVVRAFTCRKNMPTFSSW

SEQ ID NO: 18 - protein, 27 aa, artificial sequence
  hair-binding peptide
WRECRLKVAAKLRQKTLSTGNQIWIML

SEQ ID NO: 19 - protein, 27 aa, artificial sequence
  hair-binding peptide
MGHVARRWMLHTVTTTLRGLSMCLGMV

SEQ ID NO: 20 - protein, 27 aa, artificial sequence
  hair-binding peptide
FLARKLGHKGVVQVVGLDPAPHAAVRL

SEQ ID NO: 21 - protein, 27 aa, artificial sequence
  hair-binding peptide
ATSPMKRLRRASRLVKMLPRLVSWLWM

SEQ ID NO: 22 - protein, 27 aa, artificial sequence
  hair-binding peptide
HTRRQIVRPRDVFLMWCRLAKGSVKLW

SEQ ID NO: 23 - protein, 27 aa, artificial sequence
  hair-binding peptide
GKRKWLCLRSTREMVGKAIGDLGSILL

SEQ ID NO: 24 - protein, 24 aa, artificial sequence
  hair-binding peptide
ERLRVGRLQLVKGVKKIMRHLICL

SEQ ID NO: 25 - protein, 26 aa, artificial sequence
  hair-binding peptide
RVKPGKRLKTRIITCMWDSVKGPWWT

SEQ ID NO: 26 - protein, 13 aa, artificial sequence
  hair-binding peptide
SWKVWHRNRTGGP

SEQ ID NO: 27 - protein, 27 aa, artificial sequence
  hair-binding peptide
WSRWCRTNKAASMLRAWKRLGRHWEVN

SEQ ID NO: 28 - protein, 26 aa, artificial sequence
  hair-binding peptide
WRVRRLHRIVTGMCVSQYMRGSWLWT

SEQ ID NO: 29 - protein, 14 aa, artificial sequence
  hair-binding peptide
SWKVWHRNKANRVH

SEQ ID NO: 30 - protein, 27 aa, artificial sequence
  hair-binding peptide
GLLRRSAKSIISMTCKRPYLFSQLAKI

SEQ ID NO: 31 - protein, 27 aa, artificial sequence
  hair-binding peptide
HTRRQIVRPRDVFLMWCRLAKGSVKLW

SEQ ID NO: 32 - protein, 27 aa, artificial sequence
  hair-binding peptide
RRPVSFRVNKCRVRELASHGMVLWTYY

SEQ ID NO: 33 - protein, 26 aa, artificial sequence
  hair-binding peptide
LLRRAKRSALALGKVMGQYGQMWCWG

SEQ ID NO: 34 - protein, 27 aa, artificial sequence
  hair-binding peptide
FRQKCTRIRVRRQIWTYSTVGIRTWLA

SEQ ID NO: 35 - protein, 21 aa, artificial sequence
  hair-binding peptide
LRIKRVWLRVSGQVKCWFGWT

SEQ ID NO: 36 - protein, 27 aa, artificial sequence
  hair-binding peptide
VRRRGHTTRIQAYIKLARFVANGVWQL

SEQ ID NO: 37 - protein, 27 aa, artificial sequence
  hair-binding peptide
PWRRRIVWRFMRNHALASMLWLSVRTV

SEQ ID NO: 38 - protein, 27 aa, artificial sequence
  hair-binding peptide
PLWRRITKRKLVRPVATLMWYWFTSKR

SEQ ID NO: 39 - protein, 27 aa, artificial sequence
  hair-binding peptide
INRALRKLGSVLRCKSKARNTLGWAAW

SEQ ID NO: 40 - protein, 27 aa, artificial sequence
  hair-binding peptide
PWRRRIVWRFMRNHALASMLWLSVSTV

SEQ ID NO: 41 - protein, 27 aa, artificial sequence
  hair-binding peptide
ERYRLFISRLVRPILHRAWCKLTRRKY

SEQ ID NO: 42 - protein, 27 aa, artificial sequence
  hair-binding peptide
RSLRNLRWSLTRRRMTALGLYTMWALL

SEQ ID NO: 43 - protein, 29 aa, artificial sequence
  hair-binding peptide
EFGFNTTHYRDNYYLQLQCIIRSRLVRSS

SEQ ID NO: 44 - protein, 23 aa, artificial sequence
  hair-binding peptide
TRIRLLRRTKIVVRHGYKKLQCV

SEQ ID NO: 45 - protein, 28 aa, artificial sequence
  hair-binding peptide
FGFNTTHYRDNYYLQLQCIIRSRLVRSS

SEQ ID NO: 46 - protein, 27 aa, artificial sequence
  hair-binding peptide
RLRSSKTLRHVLGKALDRTKQVWCLVT

SEQ ID NO: 47 - protein, 27 aa, artificial sequence
  hair-binding peptide
RLRSSKTLQHVLGKALDRTKQVWCLVT

SEQ ID NO: 48 - protein, 27 aa, artificial sequence
  hair-binding peptide
SRIKRVILKKGLVHKIRCLVKTQFSHL

SEQ ID NO: 49 - protein, 27 aa, artificial sequence
  hair-binding peptide
RLRFSTTLCRAVHHLKRMAVKVQIVSK

SEQ ID NO: 50 - protein, 26 aa, artificial sequence
  hair-binding peptide
AVHVFRKLRRSVLWTRKVLLCTWLLR

SEQ ID NO: 51 - protein, 27 aa, artificial sequence
  hair-binding peptide
LKKRLSAILHSLACIKPVKWKWGHRKI

SEQ ID NO: 52 - protein, 27 aa, artificial sequence
  hair-binding peptide
WWKKTLSEAWCTVKKDIAGLRKVIHGH

SEQ ID NO: 53 - protein, 27 aa, artificial sequence
  hair-binding peptide
SRIKRVILKKGLVHKIRCLVETQFSHL

SEQ ID NO: 54 - protein, 21 aa, artificial sequence
  hair-binding peptide
WFVHRSHKSRKSMVRSLWCWV

SEQ ID NO: 55 - protein, 8 aa, Artificial Sequence
  Caspase 3 cleavage site
LESGDEVD

SEQ ID NO: 56 - protein, 12 aa, Artificial sequence
  Polymethylmethacrylate binding peptides
IPWWNIRAPLNA

SEQ ID NO: 57 - protein, 12 aa, Artificial Sequence
  Polymethylmethacrylate-binding peptide
TAVMNVVNNQLS

SEQ ID NO: 58 - protein, 12 aa, Artificial Sequence
  Polymethylmethacrylate-binding peptide
VPWWAPSKLSMQ

SEQ ID NO: 59 - protein, 12 aa, Artificial Sequence
  Polymethylmethacrylate-binding peptide
MVMAPHTPRARS

SEQ ID NO: 60 - protein, 12 aa, Artificial Sequence
  Polymethylmethacrylate-binding peptide
TYPNWAHLLSHY

SEQ ID NO: 61 - protein, 7 aa, Artificial Sequence
  Polymethylmethacrylate-binding peptide
TPWWRIT

SEQ ID NO: 62 - protein, 7 aa, Artificial Sequence
  Polymethylmethacrylate-binding peptide
DLTLPFH

SEQ ID NO: 63 - protein, 7 aa, Artificial Sequence
  Polymethylmethacrylate-binding peptide
GTSIPAM

SEQ ID NO: 64 - protein, 7 aa, Artificial Sequence
  Polymethylmethacrylate-binding peptide
HHKHVVA

SEQ ID NO: 65 - protein, 7 aa, Artificial Sequence
  Polymethylmethacrylate-binding peptide
HHHKHFM

SEQ ID NO: 66 - protein, 7 aa, Artificial Sequence
  Polymethylmethacrylate-binding peptide
HHHRHQG

SEQ ID NO: 67 - protein, 7 aa, Artificial Sequence
  Polymethylmethacrylate-binding peptide
HHWHAPR

SEQ ID NO: 68 - protein, 12 aa, Artificial Sequence
  Shampoo-resistant, PMMA-binding peptide
APWHLSSQYSGT

SEQ ID NO: 69 - protein, 14 aa, Artificial Sequence
  Shampoo-resistant PMMA-binding peptide
GYCLRVDEPTVCSG

SEQ ID NO: 70 - protein, 15 aa, Artificial Sequence
  Shampoo-resistant PMMA-binding peptide
HIHPSDNFPHKNRTH

SEQ ID NO: 71 - protein, 20 aa, Artificial Sequence
  Shampoo-resistant PMMA-binding peptide
HTHHDTHKPWPTDDHRNSSV

SEQ ID NO: 72 - protein, 20 aa, Artificial Sequence
  Shampoo-resistant PMMA-binding peptide
PEDRPSRTNALHHNAHHHNA

SEQ ID NO: 73 - protein, 15 aa, Artificial Sequence
  Shampoo-resistant PMMA-binding peptide
TPHNHATTNHHAGKK

SEQ ID NO: 74 - protein, 15 aa, Artificial Sequence
  Shampoo-resistant PMMA-binding peptide
EMVKDSNQRNTRISS

SEQ ID NO: 75 - protein, 12 aa, Artificial Sequence
  Shampoo-resistant PMMA-binding peptide
HYSRYNPGPHPL

SEQ ID NO: 76 - protein, 12 aa, Artificial Sequence
  Shampoo-resistant PMMA-binding peptide
IDTFYMSTMSHS

SEQ ID NO: 77 - protein, 20 aa, Artificial Sequence
  Shampoo-resistant PMMA-binding peptide
PMKEATHPVPPHKHSETPTA

SEQ ID NO: 78 - protein, 12 aa, Artificial Sequence
  Shampoo-resistant PMMA-binding peptide
YQTSSPAKQSVG

SEQ ID NO: 79 - protein, 15 aa, Artificial Sequence
  Shampoo-resistant PMMA-binding peptide
HLPSYQITQTHAQYR

SEQ ID NO: 80 - protein, 20 aa, Artificial Sequence
  Shampoo-resistant PMMA-binding peptide
TTPKTTYHQSRAPVTAMSEV

SEQ ID NO: 81 - protein, 15 aa, Artificial Sequence
  Shampoo-resistant PMMA-binding peptide
DRIHHKSHHVTTNHF

SEQ ID NO: 82 - protein, 12 aa, Artificial Sequence
  Shampoo-resistant PMMA-binding peptide
WAPEKDYMQLMK

SEQ ID NO: 83 - protein, 12 aa, Artificial sequence
  Polypropylene-binding peptides
TSDIKSRSPHHR

SEQ ID NO: 84 - protein, 12 aa, Artificial Sequence
  Polypropylenebinding peptide
HTQNMRMYEPWF

SEQ ID NO: 85 - protein, 7 aa, Artificial Sequence
  Polypropylene-binding peptide
LPPGSLA

SEQ ID NO: 86 - protein, 12 aa, Artificial Sequence
  Polypropylene-binding peptide
MPAVMSSAQVPR

SEQ ID NO: 87 - protein, 12 aa, Artificial Sequence
  Polypropylene-binding peptide
NQSFLPLDFPFR

SEQ ID NO: 88 - protein, 12 aa, Artificial Sequence
  Polypropylene-binding peptide
SILSTMSPHGAT

SEQ ID NO: 89 - protein, 12 aa, Artificial Sequence
  Polypropylenebinding peptide
SMKYSHSTAPAL

SEQ ID NO: 90 - protein, 12 aa, Artificial sequence
  Polytetrafluoroethylene-binding peptides
ESSYSWSPARLS

SEQ ID NO: 91 - protein, 12 aa, Artificial Sequence
  Polytetrafluoroethylene-binding peptide
GPLKLLHAWWQP

SEQ ID NO: 92 - protein, 7 aa, Artificial Sequence
  Polytetrafluoroethylene-binding peptide
NALTRPV

SEQ ID NO: 93 - protein, 7 aa, Artificial Sequence
  Polytetrafluoroethylene-binding peptide
SAPSSKN

SEQ ID NO: 94 - protein, 12 aa, Artificial Sequence
  Polytetrafluoroethylene-binding peptide
SVSVGMKPSPRP

SEQ ID NO: 95 - protein, 12 aa, Artificial Sequence
  Polytetrafluoroethylene-binding peptide
SYYSLPPIFHIP

SEQ ID NO: 96 - protein, 12 aa, Artificial Sequence
  Polytetrafluoroethylene-binding peptide
TFTPYSITHALL

SEQ ID NO: 97 - protein, 12 aa, Artificial Sequence
  Polytetrafluoroethylene-binding peptide
TMGFTAPRFPHY

SEQ ID NO: 98 - protein, 12 aa, Artificial Sequence
  Polytetrafluoroethylene-binding peptide
TNPFPPPPSSPA

SEQ ID NO: 99 - protein, 12 aa, Artificial sequence
  Polyethylene-binding peptides
HNKSSPLTAALP

SEQ ID NO: 100 - protein, 12 aa, Artificial Sequence
  Polyethylene-binding peptide
LPPWKHKTSGVA

SEQ ID NO: 101 - protein, 12 aa, Artificial Sequence
  Polyethylene-binding peptide
LPWWLRDSYLLP

SEQ ID NO: 102 - protein, 12 aa, Artificial Sequence
  Polyethylene-binding peptide
VPWWKHPPLPVP

SEQ ID NO: 103 - protein, 12 aa, Artificial Sequence
  Polyethylene-binding peptide
HHKQWHNHPHHA

SEQ ID NO: 104 - protein, 12 aa, Artificial Sequence
  Polyethylene-binding peptide
HIFSSWHQMWHR

SEQ ID NO: 105 - protein, 12 aa, Artificial Sequence
  Polyethylene-binding peptide
WPAWKTHPILRM

SEQ ID NO: 106 - protein, 7 aa, Artificial sequence
  Nylon-binding peptides
KTPPTRP

SEQ ID NO: 107 - protein, 7 aa, Artificial Sequence
  Nylon-binding peptide
VINPNLD

SEQ ID NO: 108 - protein, 7 aa, Artificial Sequence
  Nylon-binding peptide
KVWIVST

SEQ ID NO: 109 - protein, 7 aa, Artificial Sequence
  Nylon-binding peptide
AEPVAML

SEQ ID NO: 110 - protein, 7 aa, Artificial Sequence
  Nylon-binding peptide
AELVAML

SEQ ID NO: 111 - protein, 7 aa, Artificial Sequence
  Nylon-binding peptide
HSLRLDW

SEQ ID NO: 112 - protein, 13 aa, Artificial sequence
  Polystyrene-binding peptide
TSTASPTMQSKIR

SEQ ID NO: 113 - protein, 12 aa, Artificial Sequence
  Polystyrene-binding peptide
KRNHWQRMHLSA

SEQ ID NO: 114 - protein, 12 aa, Artificial Sequence
  Polystyrene-binding peptide
SHATPPQGLGPQ

SEQ ID NO: 115 - protein, 20 aa, artificial sequence
  cellulose acetate-binding peptide
ATTPPSGKAAAHSAARQKGN

SEQ ID NO: 116 - protein, 15 aa, artificial sequence
  cellulose acetate-binding peptide
DTIHPNKMKSPSSPL

SEQ ID NO: 117 - protein, 20 aa, artificial sequence
  cellulose aceteate-binding peptide
NGNNHTDIPNRSSYTGGSFA

SEQ ID NO: 118 - protein, 15 aa, ARTIFICIAL SEQUENCE
  synthetic cellulose acetate-binding peptide
SDETGPQIPHRRPTW

SEQ ID NO: 119 - protein, 7 aa, Artificial Sequence
  Pigment-binding peptide
MPPPLMQ

SEQ ID NO: 120 - protein, 7 aa, Artificial Sequence
  Pigment-binding peptide
FHENWPS

SEQ ID NO: 121 - protein, 12 aa, Artificial Sequence
  Pigment-binding peptide
RTAPTTPLLLSL

SEQ ID NO: 122 - protein, 12 aa, Artificial Sequence
  Pigment-binding peptide
WHLSWSPVPLPT

SEQ ID NO: 123 - protein, 7 aa, Artificial Sequence
  Pigment-binding peptide
PHARLVG

SEQ ID NO: 124 - protein, 7 aa, Artificial Sequence
  Pigment-binding peptide
NIPYHHP

SEQ ID NO: 125 - protein, 7 aa, Artificial Sequence
  Pigment-binding peptide
TTMPAIP

SEQ ID NO: 126 - protein, 7 aa, Artificial Sequence
  Pigment-binding peptide
HNLPPRS

SEQ ID NO: 127 - protein, 12 aa, Artificial Sequence
  Pigment-binding peptide
AHKTQMGVRQPA

SEQ ID NO: 128 - protein, 12 aa, Artificial Sequence
  Pigment-binding peptide
ADNVQMGVSHTP

SEQ ID NO: 129 - protein, 12 aa, Artificial Sequence
  Pigment-binding peptide
AHNAQMGVSHPP

SEQ ID NO: 130 - protein, 12 aa, Artificial Sequence
  Pigment-binding peptide
ADYVGMGVSHRP

SEQ ID NO: 131 - protein, 12 aa, Artificial Sequence
  Pigment-binding peptide
SVSVGMKPSPRP

SEQ ID NO: 132 - protein, 7 aa, Artificial Sequence
  Pigment-binding peptide
YPNTALV

SEQ ID NO: 133 - protein, 7 aa, Artificial Sequence
  Pigment-binding peptide
VATRIVS

SEQ ID NO: 134 - protein, 12 aa, Artificial Sequence
  Pigment-binding peptide
HSLKNSMLTVMA

SEQ ID NO: 135 - protein, 7 aa, Artificial Sequence
  Pigment-binding peptide
NYPTQAP

SEQ ID NO: 136 - protein, 7 aa, Artificial Sequence
  Pigment-binding peptide
KCCYSVG

SEQ ID NO: 137 - protein, 12 aa, Artificial Sequence
  Pigment-binding peptide
RHDLNTWLPPVK

SEQ ID NO: 138 - protein, 12 aa, Pigment-binding peptide
EISLPAKLPSAS

SEQ ID NO: 139 - protein, 12 aa, Artificial Sequence
  Pigment-binding peptide
SDYVGMRPSPRH

SEQ ID NO: 140 - protein, 12 aa, Artificial Sequence
  Pigment-binding peptide
SDYVGMRLSPSQ

SEQ ID NO: 141 - protein, 12 aa, Artificial Sequence
  Pigment-binding peptide
SVSVGIQPSPRP

SEQ ID NO: 142 - protein, 12 aa, Artificial Sequence
  Pigment-binding peptide
YVSVGIKPSPRP

SEQ ID NO: 143 - protein, 12 aa, Artificial Sequence
  Pigment-binding peptide
YVCEGIHPCPRP

SEQ ID NO: 144 - protein, 12 aa, artificial sequence
  synthetic pigment binding peptide
WAPEKDHMQLMK

SEQ ID NO: 145 - protein, 12 aa, artificial sequence
  Iron oxide-binding peptide
WAPEKDYMQLMK

SEQ ID NO: 146 - protein, 20 aa, artificial sequence
  Iron oxide-binding peptide
CPLDTPTHKTKHEYKTRCRH

SEQ ID NO: 147 - protein, 18 aa, artificial sequence
  Iron oxide-binding peptide
DHDHPRLHKRQEKSEHLH

SEQ ID NO: 148 - protein, 20 aa, artificial sequence
  Iron oxide-binding peptide
DSHHNHHKQDSRPQHRKTPN

SEQ ID NO: 149 - protein, 15 aa, artificial sequence
  Iron oxide-binding peptide
EGGNAPHHKPHHRKH

SEQ ID NO: 150 - protein, 18 aa, artificial sequence
  Iron oxide-binding peptide
HDSHRPLTQHGHRHSHVP

SEQ ID NO: 151 - protein, 18 aa, artificial sequence
  Iron oxide-binding peptide
HDSNHCSHSTRRPNCART

SEQ ID NO: 152 - protein, 15 aa, artificial sequence
  Iron oxide-binding peptide
ATRVDNTPASNPPSL

SEQ ID NO: 153 - protein, 15 aa, artificial sequence
  Iron oxide-binding peptide
DGIKPFHLMTPTLAN

SEQ ID NO: 154 - protein, 18 aa, artificial sequence
  Iron oxide-binding peptide
DITPPGSTHHRKPHRHQH

SEQ ID NO: 155 - protein, 15 aa, artificial sequence
  Iron oxide-binding peptide
DNLWPQPLNVEDDRY

SEQ ID NO: 156 - protein, 18 aa, artificial sequence
  Iron oxide-binding peptide
ENEKHRHNTHEALHSHFK

SEQ ID NO: 157 - protein, 20 aa, artificial sequence
  Iron oxide-binding peptide
GAIWPASSALMTEHNPTDNH

SEQ ID NO: 158 - protein, 15 aa, artificial sequence
  Iron oxide-binding peptide
GDTNQDTVMWYYTVN

SEQ ID NO: 159 - protein, 15 aa, artificial sequence
  Iron oxide-binding peptide
HNGPYGMLSTGKIHF

SEQ ID NO: 160 - protein, 15 aa, artificial sequence
  Iron oxide-binding peptide
LDGGYRDTPDNYLKG

SEQ ID NO: 161 - protein, 15 aa, artificial sequence
  Iron oxide-binding peptide
LHTKTENSHTNMKTT

SEQ ID NO: 162 - protein, 20 aa, artificial sequence
  Iron oxide-binding peptide
NAQYDPPTLNKGAVRKAAST

SEQ ID NO: 163 - protein, 15 aa, artificial sequence
  Iron oxide-binding peptide
NGNNHTDIPNRSSYT

SEQ ID NO: 164 - protein, 18 aa, artificial sequence
  Iron oxide-binding peptide
QSTNHHHPHAKHPRVNTH

SEQ ID NO: 165 - protein, 15 aa, artificial sequence
  Iron oxide-binding peptide
SNNDYVGTYPATAIQ

SEQ ID NO: 166 - protein, 15 aa, artificial sequence
  Iron oxide-binding peptide
STQHNLHDRNIYFVS

SEQ ID NO: 167 - protein, 20 aa, artificial sequence
  Iron oxide-binding peptide
TANNKTPAGAPNAAVGLAQR

SEQ ID NO: 168 - protein, 15 aa, artificial sequence
  Iron oxide-binding peptide
TEPTRISNYRSIPND

SEQ ID NO: 169 - protein, 18 aa, artificial sequence
  Iron oxide-binding peptide
THNPREHARHHHHNEYKH

SEQ ID NO: 170 - protein, 15 aa, artificial sequence
  Iron oxide-binding peptide
THPPCWYETNCIVQE

SEQ ID NO: 171 - protein, 20 aa, artificial sequence
  Iron oxide-binding peptide
TTNPHKPASHHHDHRPALRH

SEQ ID NO: 172 - protein, 15 aa, artificial sequence
  Iron oxide-binding peptide
WLVADNATDGHSHQK

SEQ ID NO: 173 - protein, 15 aa, artificial sequence
  Iron oxide-binding peptide
YTDSMSDQTPEFAKY

SEQ ID NO: 174 - protein, 27 aa, artificial sequence
  Clay-binding peptide
GHGSPSNSHHGSKKCDMGNSRAKCKRL

SEQ ID NO: 175 - protein, 27 aa, artificial sequence
  Clay-binding peptide
SDRHNLRNSWSISRHCRRKQGRCLPAH

SEQ ID NO: 176 - protein, 27 aa, artificial sequence
  Clay-binding peptide
KKSNKGHHPSSKGKGPPWSEWDKKNGP

SEQ ID NO: 177 - protein, 27 aa, artificial sequence
  Clay-binding peptide
KKSNKGPHPSSKGKGPPWSEWDKKNGP

SEQ ID NO: 178 - protein, 22 aa, artificial sequence
  Clay-binding peptide
VGRHHSKAKQKRPHGGKGQNKN

SEQ ID NO: 179 - protein, 22 aa, artificial sequence
  Clay-binding peptide
VGRHHPKAKQKRPHGGKGQNKN

SEQ ID NO: 180 - protein, 17 aa, artificial sequence
  Clay-binding peptide
GRRPRARGRSRRGSTKT

SEQ ID NO: 181 - protein, 19 aa, artificial sequence
  Clay-binding peptide
LGVIRNHVVRGRRHHQHVR

SEQ ID NO: 182 - protein, 27 aa, artificial sequence
  Clay-binding peptide
QPGRPTEVHPELVRKSAYLVNPSEDIR

SEQ ID NO: 183 - protein, 27 aa, artificial sequence
  Clay-binding peptide
HRSEKPKNVKYKRGYWERGNQKKHGPG

SEQ ID NO: 184 - protein, 27 aa, artificial sequence
  Clay-binding peptide
GSHKRRGSYALLRTRGVGRQAELEHLL

SEQ ID NO: 185 - protein, 27 aa, artificial sequence
  Clay-binding peptide
VGEKPRRKSKGAKAKKARTKEEKLPKN

SEQ ID NO: 186 - protein, 27 aa, artificial sequence
  Clay-binding peptide
NKGHKQSGSPRHSNKKEKKTQQKRGQP

SEQ ID NO: 187 - protein, 27 aa, artificial sequence
  Clay-binding peptide
HWGSQHKTGLRNHKRSRRDSLGKRGTD

SEQ ID NO: 188 - protein, 27 aa, artificial sequence
  Clay-binding peptide
KGWGSSSGPPGLTGKALGKGRLKPKKK

SEQ ID NO: 189 - protein, 27 aa, artificial sequence
  Calcium carbonate binding peptide
RNNKGSKKVDDKRRKTVHNTKSRAKYS

SEQ ID NO: 190 - protein, 27 aa, artificial sequence
  Calcium carbonate binding peptide
RNNKGSKKVDDKRRKTVHNTKSRAKHS

SEQ ID NO: 191 - protein, 27 aa, artificial sequence
  Calcium carbonate binding peptide
RDNKGSKKVDDKRRKTVHNTKSRAKYS

SEQ ID NO: 192 - protein, 27 aa, artificial sequence
  Calcium carbonate binding peptide
RNNKGSKKVDDKRRKTVHSTKSRAKYS

SEQ ID NO: 193 - protein, 27 aa, artificial sequence
  Calcium carbonate binding peptide
RNNKGSRKVDDKRRKTVHNTKSRAKYS

SEQ ID NO: 194 - protein, 27 aa, artificial sequence
  Calcium carbonate binding peptide
RNNKGSKKADDKRRKTVHSTKSRAKYS

SEQ ID NO: 195 - protein, 27 aa, artificial sequence
  Calcium carbonate binding peptide
RNNKGSKKVDDKRRKAVHNKKSRAKYS

SEQ ID NO: 196 - protein, 27 aa, artificial sequence
  Calcium carbonate binding peptide
RNNKGSKKVDDKRRKTVHNTRSRAKYS

SEQ ID NO: 197 - protein, 27 aa, artificial sequence
  Calcium carbonate binding peptide
RNNKGSKKVDDKRRKTVHNTKSRAKFS

SEQ ID NO: 198 - protein, 27 aa, artificial sequence
  Calcium carbonate binding peptide
QRRKLRHPKEKWFGWSEKKVIKKWSRK

SEQ ID NO: 199 - protein, 27 aa, artificial sequence
  Calcium carbonate binding peptide
QRRKFRHPKEKWFGWSEKKVIKXNGRP

SEQ ID NO: 200 - protein, 27 aa, artificial sequence
  Calcium carbonate binding peptide
HKRLVQNKPHRTRKIEGWIKHMVKRQH

SEQ ID NO: 201 - protein, 27 aa, artificial sequence
  Calcium carbonate binding peptide
TRGHIMRPCWIGAMKQGVKKKRTPGWR

SEQ ID NO: 202 - protein, 27 aa, artificial sequence
  Calcium carbonate binding peptide
WKVKRRMVTRTYEFMGKKPCMMLTKRL

SEQ ID NO: 203 - protein, 26 aa, artificial sequence
  Calcium carbonate binding peptide
KKSNKGHHSKAKQKRPHGGKAQNKNT

SEQ ID NO: 204 - protein, 27 aa, artificial sequence
  Calcium carbonate binding peptide
RAHKERFVVRQIGRSQGYKTWQCVRVA

SEQ ID NO: 205 - protein, 27 aa, artificial sequence
  Calcium carbonate binding peptide
SQKPKGHKVKVVVKLCKRPYWRMLNTA

SEQ ID NO: 206 - protein, 27 aa, artificial sequence
  Calcium carbonate binding peptide
NHGCPVNWKVXNPPRGWQRLNHCKWWN

SEQ ID NO: 207 - protein, 27 aa, artificial sequence
  Calcium carbonate binding peptide
RNSRHKEWRRYKRTHVHSHEFYHVECW

SEQ ID NO: 208 - protein, 27 aa, artificial sequence
  Calcium carbonate binding peptide
HRSEKPKNVNYKRGYWERGNQKKHGPG

SEQ ID NO: 209 - protein, 27 aa, artificial sequence
  Calcium carbonate binding peptide
HERTRRGKPDRQKTTHEKRRQGLWIFM

SEQ ID NO: 210 - protein, 27 aa, artificial sequence
  Calcium carbonate binding peptide
PWGTNKRQKHKVHEAKALKKSLWYSNS

SEQ ID NO: 211 - protein, 27 aa, artificial sequence
  Calcium carbonate binding peptide
RRGVVLCHTHRNKRIRLAYSVTKKAWA

SEQ ID NO: 212 - protein, 27 aa, artificial sequence
  Calcium carbonate binding peptide
ERIRWRRLSAEIRAHKWSVLKFRLSCM

SEQ ID NO: 213 - protein, 27 aa, artificial sequence
  Calcium carbonate binding peptide
KTKEKKKEVKLHKKSLSLVLLADLWRL

SEQ ID NO: 214 - protein, 27 aa, artificial sequence
  Calcium carbonate binding peptide
LGKKHKQHSKVGHGKLSTRFLRRSKLF

SEQ ID NO: 215 - protein, 20 aa, artificial sequence
  Silica-binding peptide
AEAKRHPVVPLHEQHGHHEL

SEQ ID NO: 216 - protein, 18 aa, artificial sequence
  Silica-binding peptide
APQTWNRPHPGHPNVHTR

SEQ ID NO: 217 - protein, 20 aa, artificial sequence
  Silica-binding peptide
ATTPPSGKAAAHSAARQKGN

SEQ ID NO: 218 - protein, 20 aa, artificial sequence
  Silica-binding peptide
DGRPDNPKHQQSYNRQLPRQ

SEQ ID NO: 219 - protein, 20 aa, artificial sequence
  Silica-binding peptide
DHNNRQHAVEVRENKTHTAR

SEQ ID NO: 220 - protein, 20 aa, artificial sequence
  Silica-binding peptide
GPEPRALNPKRHMDPATQIR

SEQ ID NO: 221 - protein, 15 aa, artificial sequence
  Silica-binding peptide
HDHHQTHNVLHGMKK

SEQ ID NO: 222 - protein, 18 aa, artificial sequence
  Silica-binding peptide
HHDRAEPRGMAATLAQTI

SEQ ID NO: 223 - protein, 20 aa, artificial sequence
  Silica-binding peptide
HHNHMTGADNPIFHNNTAHR

SEQ ID NO: 224 - protein, 18 aa, artificial sequence
  Silica-binding peptide
HNHAQMLRPEPTGISHKN

SEQ ID NO: 225 - protein, 20 aa, artificial sequence
  Silica-binding peptide
HTNDNGQSTPRRDPPAFQRK

SEQ ID NO: 226 - protein, 18 aa, artificial sequence
  Silica-binding peptide
HTNHHYDQKMHGPLPTPY

SEQ ID NO: 227 - protein, 20 aa, artificial sequence
  Silica-binding peptide
LNSMSDKHHGHQNTATRNQH

SEQ ID NO: 228 - protein, 20 aa, artificial sequence
  Silica-binding peptide
MHKPNNPDTHRSTPSPLGKS

SEQ ID NO: 229 - protein, 18 aa, artificial sequence
  Silica-binding peptide
NFPVYDTTHHGGHRSKLH

SEQ ID NO: 230 - protein, 20 aa, artificial sequence
  Silica-binding peptide
NVHPQSENTNTTRPHKSTQR

SEQ ID NO: 231 - protein, 18 aa, artificial sequence
  Silica-binding peptide
QHGMHSPNLGARMNATPH

SEQ ID NO: 232 - protein, 20 aa, artificial sequence
  Silica-binding peptide
RPNDTHHPGKCDTHAVCHQT

SEQ ID NO: 233 - protein, 20 aa, artificial sequence
  Silica-binding peptide
SHLMHVKAPTDQASTRNRFD

SEQ ID NO: 234 - protein, 20 aa, artificial sequence
  Silica-binding peptide
SSSTPPNSPKHSKYNVWTSP

SEQ ID NO: 235 - protein, 17 aa, artificial sequence
  Silica-binding peptide
VHQTTPQHKDAVNLPRK

SEQ ID NO: 236 - protein, 20 aa, artificial sequence
  Silica-binding peptide
WHSSEGQYKKPNNHRQYHTG

SEQ ID NO: 237 - protein, 15 aa, artificial sequence
  Silica-binding peptide
YKHERHYSQPLKVRH

SEQ ID NO: 238 - protein, 17 aa, Artificial Sequence
  synthetic construct
PKGLKKLLKGLKKLLKL

SEQ ID NO: 239 - protein, 16 aa, Artificial Sequence
  synthetic construct
KGLKKLLKGLKKLLKL

SEQ ID NO: 240 - protein, 16 aa, Artificial Sequence
  synthetic construct
KGLKKLLKLLKKLLKL

SEQ ID NO: 241 - protein, 14 aa, Artificial Sequence
  synthetic construct
LKKLLKLLKKLLKL

SEQ ID NO: 242 - protein, 12 aa, Artificial Sequence
  synthetic construct
LKKLLKLLKKLL

SEQ ID NO: 243 - protein, 17 aa, Artificial Sequence
  synthetic construct
VAKKLAKLAKKLAKLAL

SEQ ID NO: 244 - protein, 13 aa, Artificial Sequence
  synthetic construct
FAKLLAKALKKLL

SEQ ID NO: 245 - protein, 16 aa, Artificial Sequence
  synthetic construct
KGLKKGLKLLKKLLKL

SEQ ID NO: 246 - protein, 16 aa, Artificial Sequence
  synthetic construct
KGLKKLLKLGKKLLKL

SEQ ID NO: 247 - protein, 16 aa, Artificial Sequence
  synthetic construct
KGLKKLGKLLKKLLKL

SEQ ID NO: 248 - protein, 16 aa, Artificial Sequence
  synthetic construct
KGLKKLLKLLKKGLKL

SEQ ID NO: 249 - protein, 16 aa, Artificial Sequence
  synthetic construct
KGLKKLLKLLKKLGKL

SEQ ID NO: 250 - protein, 19 aa, Artificial Sequence
  synthetic construct
FALALKALKKLKKALKKAL

SEQ ID NO: 251 - protein, 17 aa, Artificial Sequence
  synthetic construct
FAKKLAKLAKKLAKLAL

SEQ ID NO: 252 - protein, 13 aa, Artificial Sequence
  synthetic construct
FAKLLAKLAKKLL

SEQ ID NO: 253 - protein, 15 aa, Artificial Sequence
  synthetic construct
FAKKLAKLALKLAKL

SEQ ID NO: 254 - protein, 10 aa, Artificial Sequence
  synthetic construct
FAKKLAKKLL

SEQ ID NO: 255 - protein, 13 aa, Artificial Sequence
  synthetic construct
FAKLLAKLAKKVL

SEQ ID NO: 256 - protein, 13 aa, Artificial Sequence
  synthetic construct
KYKKALKKLAKLL

SEQ ID NO: 257 - protein, 12 aa, Artificial Sequence
  synthetic construct
FALLKALLKKAL

SEQ ID NO: 258 - protein, 14 aa, Artificial Sequence
  synthetic construct
KRLFKKLKFSLRKY

SEQ ID NO: 259 - protein, 14 aa, Artificial Sequence
  synthetic construct
KRLFKKLLFSLRKY

SEQ ID NO: 260 - protein, 14 aa, Artificial Sequence
  synthetic construct
LLLFLLKKRKKRKY

SEQ ID NO: 261 - protein, 36 aa, H. cecropia
KWKLFKKIEKVGQNIRDGIIKAGPAVAWGQATQIAK

SEQ ID NO: 262 - protein, 23 aa, Xenopus
GIGKFLHSAKKFGKAFVGEIMNS

SEQ ID NO: 263 - protein, 22 aa, Xenopus
GIGKFLKKAKKFGKAFVKILKK

SEQ ID NO: 264 - protein, 12 aa, Bos taurus
RLCRIVVIRVCR

SEQ ID NO: 265 - protein, 13 aa, Bos sp.
ILPWKWPWWPWRR

SEQ ID NO: 266 - protein, 24 aa, Homo sapiens
DSHAKRHHGYKRKFHEKHHSHRGY

SEQ ID NO: 267 - protein, 33 aa, artificial sequence
  synthetic linker
EPEPEPEPIPEPPKEAPVVIEKPKPKPKPKPKP

SEQ ID NO: 268 - protein, 21 aa, artificial sequence
  synthetic peptide linker
GKGKGKGKGKGKGKGKGKGKG

SEQ ID NO: 269 - protein, 8 aa, artificial sequence
  synthetic peptide bridge
GSGGGGSP

SEQ ID NO: 270 - protein, 16 aa, artificial sequence
  synthetic peptide bridge
GSGGGGSPGSGGGGSP

SEQ ID NO: 271 - protein, 28 aa, artificial sequence
  hair-binding peptide
PLWRRITKRKLVRPVATLMWYWFTSKRK